(19) United States
(12) Patent Application Publication
(10) Pub. No.: US 2025/0312498 A1
(43) Pub. Date: Oct. 9, 2025

(54) PROTOCOL FOR MINIMIZING TOXICITY OF COMBINATION DOSAGES AND IMAGING AGENT FOR VERIFICATION

(71) Applicant: ProLynx LLC, San Francisco, CA (US)

(72) Inventors: Brian HEARN, Moraga, CA (US); Daniel V. Santi, San Francisco, CA (US); Shaun Fontaine, Conord, CA (US); Gary W. Ashley, Alameda, CA (US)

(73) Assignee: ProLynx LLC, San Francisco, CA (US)

(21) Appl. No.: 19/088,784

(22) Filed: Mar. 24, 2025

Related U.S. Application Data
(63) Continuation of application No. 18/118,650, filed on Mar. 7, 2023, now abandoned.
Application No. 18/118,650 is a continuation of application No. 16/961,633, filed on Jul. 10, 2020, now abandoned.
(60) Provisional application No. 62/674,483, filed on May 21, 2018.
(60) Provisional application No. 62/700,147, filed on Jul. 18, 2018.
(60) Provisional application No. 62/711,421, filed on Jul. 27, 2018.
(60) Provisional application No. 62/711,423, filed on Jul. 27, 2018.
(60) Provisional application No. 62/716,788, filed on Aug. 9, 2018.
(60) Provisional application No. 62/716,796, filed on Aug. 9, 2018.

(51) Int. Cl. A61K 51/06; A61K 9/51; A61K 31/4745; A61K 45/06; A61K 47/60; A61P 35/00; B82Y 5/00

(52) U.S. Cl. CPC A61K 51/065; A61K 9/51; A61K 31/4745; A61K 47/60; A61P 35/00; A61K 45/06; B82Y 5/00

(57) ABSTRACT

Advantage is taken of the enhanced permeability and retention effect (EPR effect) to shield normal tissue from exposure to combinations of chemotherapeutic agents. Imaging agents that exhibit the enhanced permeability and retention (EPR) effect in solid tumors are useful in mimicking the behavior of chemotherapeutic or other drugs for treatment of said tumor conjugated to carriers of similar size and shape to the carriers of said imaging agents.

### CROSS-REFERENCE TO RELATED APPLICATIONS

This application is a continuation of U.S. patent application Ser. No. 18/118,650, filed Mar. 7, 2023, which is a continuation of U.S. patent application Ser. No. 16/961,633, having an international filing date of Jan. 11, 2019, which is a national stage application under 35 U.S.C. § 371 of International Application No. PCT/US2019/013306, filed internationally on Jan. 11, 2019, which claims priority from U.S. provisional application 62/617,095 filed 12 Jan. 2018, U.S. provisional application 62/674,483 filed 21 May 2018, U.S. provisional application 62/711,421 filed 27 Jul. 2018, U.S. provisional application 62/716,788 filed 9 Aug. 2018, U.S. provisional application 62/716,796 filed 9 Aug. 2018, U.S. provisional application 62/700,147 filed 18 Jul. 2018, and U.S. provisional application 62/711,423 filed 27 Jul. 2018, the disclosures of which are herein incorporated by reference in their entirety.

### TECHNICAL FIELD

The invention is in the field of combination treatments of solid tumors and of diagnostic methods that assess pharmacokinetics of administered entities, specifically with respect to the enhanced permeability and retention (EPR) effect exhibited when entities of nanometer dimensions are administered to subjects with solid tumors. More specifically, the invention relates to taking advantage of the EPR effect exhibited when conjugates of nanometer dimensions are administered to subjects with solid tumors.

### BACKGROUND ART

Chemotherapeutic agents that are used to treat solid tumors are toxic to normal tissue as well. Levels of such agents administered are limited by their maximum tolerated dose. When combinations of such agents are used, the toxicity of both agents is experienced by normal tissue which further limits effective dosage levels. This problem has been addressed by designing protocols that avoid simultaneous administration of more than one agent essentially on a trial-and-error basis which does not lead to optimal results. Another approach has been to utilize synergistic combinations of two or more agents where their synergistic ratio is maintained by controlling the pharmacokinetics using suitable delivery vehicles, as set forth in U.S. Pat. Nos. 7,850,990 and 9,271,931. Since the drugs are acting in synergy, lower dosage levels are effective, thus also ameliorating the inherent toxicity of the drugs.

Despite these approaches in the art, there remains a need for successful design of protocols that will minimize the toxic effect of drug combinations on normal tissue. The present invention solves this problem by taking advantage of the enhanced permeability and retention effect (EPR) of large molecules that can be used as carriers in order to control exposure of normal tissue to the toxic drug and, by virtue of the present invention, assuring that the EPR effect is shown by these conjugates.

As early as 1986, Maeda and coworkers demonstrated an EPR effect in solid tumors (Matsumura, Y., and Maeda, H., Cancer Res. (1986) 46:6387-6392). Later work by this same group confirms this effect (Maeda, H., et al., J. Controlled Release (2001) 74:47-61; Maeda, H., et al., Eur. J. Pharm. Biopharm. (2009) 71:409-419). Essentially, these authors showed that solid tumors growing beyond the size of a few millimeters in diameter depend on neovasculature that differs from normal vasculature in its architecture. While the cutoff pore size of normal vasculature is in the range of 2-6 nm, the neovasculature in solid tumors has a pore cutoff range of 100-700 nm (Dreher, M. R., et al., J. Natl. Cancer Inst. (2006) 98:335-344; Singh, Y., et al., Molecular Pharmaceutics (2012) 9:144-155). The larger pores in the tumor neovasculature result in leakiness that allows macromolecules and nanoparticles to penetrate and extravasate into the tumor and this combined with poor lymphatic drainage results in the EPR effect which results in accumulation of macromolecules, conjugates or nanoparticles that is generally related to size and flexibility of the nanoparticle or macromolecule and exposure (i.e., t_{1/2}). This has in particular been demonstrated for liposomal delivery as noted, for example, by Allen, T., et al., Science (2004) 303:1818-1822. Useful reviews of the literature describing this effect include Danhier, F., et al., J. Control Rel. (2010) 148:135-146 and Eshun, F. K., et al., Gene Ther. (2010) 17:922-929. With various size dextrans, it has been shown that there is an optimal size of ˜40- to 60 kDa and t_{1/2} (exposure time) that provides the most accumulation by the EPR effect (Dreher, M. R., et al. J Natl Cancer Inst (2006) Supra.)

In one aspect, the present invention relies on taking advantage of the EPR effect even for small molecules by providing conjugates to nanomolecular carriers, especially flexible carriers and by permitting determination of the pharmacokinetics associated with the EPR effect by providing an imaging agent coupled to a carrier of similar dimensions to those of a carrier used to deliver small molecules administered as conjugates to nanomolecular carriers, especially flexible carriers.

Jain, et al., have described features of the EPR effect relevant to nanomedicine design (Chauhan, V. P., and Jain, R. K., Nat. Mater. (2013) 12:958-962; Chauhan, V. P., et al., Angew. Chem. Int. Ed. Engl. (2011) 50:11417-11420; Chauhan, V. P., et al., Nat. Nanotechnol. (2012) 7:383-388). Tumor vessel walls and tissue matrix exist as a series of inter-connected pores with variable cross-sections. Cutoff sizes only indicate the largest particle that penetrates, and large particles generally penetrate tumors heterogeneously and suboptimally compared with smaller particles. The vascular pore-size distribution within a single tumor can vary by orders of magnitude, with most of the pores actually being much smaller than the pore cutoff size. Thus, the effective vascular permeability of small particles does not necessarily correlate with cutoff size; smaller particles penetrate tumors more rapidly and uniformly than larger particles and smaller particles carrying drugs should be more generally effective against solid tumors than larger particles.

The shape of the nanoparticles also modifies the EPR effect (Chauhan, V. P., (2011) supra). Non-spherical nanoparticles can penetrate tumors more rapidly and accumulate at higher levels than size-matched spheres, because of enhanced penetration through the pores is related to the shortest dimension of the particle. The advantage of non-spherical particles holds for smaller vessel-pore-sizes but is lost with respect to large pore sizes.

Many or most studies of nanoparticles for EPR drug delivery and imaging utilize larger ˜100 nm liposomes/particles containing appropriate drugs or isotopes. As described above, regardless of cut-off pore size these larger nanoparticles are likely not the optimal size for accumulation in many tumors since most will contain neovasculature with heterogeneous pore sizes; thus the present invention is focused on carriers with hydrodynamic radii of less than 50 nm.

The present invention, in some embodiments, employs linking technologies that are particularly favorable for preparation of conjugates designed to take advantage of the EPR effect. In particular, linkages that release a small molecule chemotherapeutic agent (drug) by beta elimination have been disclosed. See, for example, U.S. Pat. Nos. 8,680,315; 9,387,254; 8,754,190; 8,946,405; and 8,703,907, and WO 2015/051307, all incorporated herein by reference. Such linkers permit tuning of the time of release of the coupled drug by adjusting the acidity of a carbon-hydrogen bond positioned beta to a suitable leaving group.

It has also been possible to study this effect by using detectable markers coupled to nanoparticles. Wilks, M. Q., et al. (Bioconjug. Chem. (2015) 26:1061-1069) reported a 30 kDa PEG-DFB-⁸⁹Zr conjugate (also containing fluorescent Cy5.5). In the mouse, it showed an elimination t_{1/2} of 13.5 hr and high retention (˜4 to 5% ID/g) in an implanted HT-29 tumor at 48 hr post injection. The kinetics of tumor accumulation, clearance or capacity were not determined. Because these nanoparticles are only about 10 nm and are flexible, their biological distribution does not show a strong EPR effect in tumor tissue. However, this study shows that labeled conjugates can be thus used to elucidate these parameters.

Another technology useful in the method of the invention is positron emission tomography (PET) which offers some advantages over the use of fluorescent label for such studies. Current knowledge on the EPR effect in human tumors is largely based on studies of low-resolution single photon imaging techniques of radiolabeled liposomes c.f. (Harrington, K. J., et al., Clin. Cancer Res. (2001) 7:243-254; Khalifa, A. et al., Nucl. Med. Commun. (1997) 18:17-23), which could visualize tumors but could not quantitate the EPR effect. The high detection sensitivity/quantitation and spatial resolution of PET make this technology superior for quantitative studies of nanoparticle biodistribution. For example, Lee H, et al., Clin Cancer Res 23(15):4190-4202, showed that 64Cu-labeled HER2-targeted liposomal doxorubicin—about/over 100 nm diameter—accumulated in human tumors and could be quantified by PET. The range of intra- and inter-patient tumor drug concentrations measured was proposed to result in variable antitumor activity of these liposomes that included both a therapeutic and diagnostic (PET labeled) moieties, designated herein theranostic nanoparticles (TNP). Tumor deposition was stratified and uptake levels were retrospectively associated with treatment outcomes: high uptake tumors were susceptible to the effect of the TNPs (75% partial remission/stable disease) whereas low-uptake tumors (43% stable disease) were not. Brain metastases were also imaged, suggesting their vasculature had increased pore sizes that could make such metastasis susceptible to TNPs. These results indicate that a NP imaging approach may be applicable as a predictive strategy for personalizing nanomedicines, whereby a diagnostic procedure is performed, and then only patients with susceptible tumors are treated with the TNPs. In summary, these data suggest that it may be possible to use pretreatment imaging of NP deposition in tumors to identify patients most likely to benefit from treatment with closely related TNPs.

Using these tools available in the art, protocols are constructed that ameliorate the toxic effect of combination therapy on normal tissue.

### DISCLOSURE OF THE INVENTION

One goal of the invention is to confine the cytotoxic effect of drugs administered in combination to tumor tissue while sparing normal tissue to the extent possible. In one approach, this can be done by adjusting the dosage administration protocol so that while a first chemotherapeutic agent is sequestered in a solid tumor and no longer available in the system to exert an effect on normal tissue a second therapeutic agent is administered so that effectively only the toxic effects of the second drug, without supplementation by the first, are exerted in the system while the combined effects are exerted in the tumor. In a second approach, both agents are sequestered as conjugates in the solid tumor so that higher concentrations of both agents are experienced by tumor cells than are experienced by normal tissue and the agents are cleared from normal tissue while remaining in the tumor.

Thus, in one aspect, the invention is directed to a method to ameliorate the toxicity to normal tissue in a subject resulting from administering to said subject a first and second chemotherapeutic agent in a protocol for combination therapy against a solid tumor employing said first and second agent, which method comprises:
administering the first agent as an agent-releasing conjugate to a carrier, wherein the carrier is a nanoparticle or macromolecule each with a hydrodynamic radius of 5-50 nm (i.e., a diameter of 10-100 nm) which conjugate exhibits enhanced permeability and retention (EPR) in solid tumors so as to concentrate said conjugate in the tumor and wherein the rate of release from the tumor of the conjugate and first agent released from the conjugate is substantially slower than the rate of clearance of the conjugate and released agent from the systemic circulation of the subject;
allowing a time period for clearance of the conjugate and released agent from the systemic circulation of the subject; and
after said time period, administering said second agent to the subject.

In some embodiments, an additional agent that has a non-overlapping toxicity with the second agent may also be administered.

In a second aspect, the invention is directed to a method to minimize the toxic effects on normal tissue of a subject of a first and second chemotherapeutic agent used in combination to treat a solid tumor in said subject which method comprises administering both said first and second agents as releasable conjugates to carriers, wherein the carriers are nanoparticles or macromolecules each with a hydrodynamic radius of 5-50 nm (10-100 nm diameter) wherein said conjugates exhibit enhanced permeability and retention (EPR) and effect concentration of both said conjugates in said tumor.

In some embodiments of the simultaneous administration, only the first agent is conjugated and the second agent is in unconjugated form.

In some instances, a third similarly conjugated or unconjugated therapeutic agent may be employed as well.

In connection with the foregoing methods, when the second or third agent is conjugated the carriers mimic those of the first agent. In any case, labeled non-releasable conjugates comprising carriers with the same characteristics as those used in conjugating the drugs can be used to monitor the uptake of the conjugates by the solid tumor. Administering such conjugate where the carrier is non-releasably linked to the label permits verification (or not) that the corresponding conjugates of drugs will exhibit an EPR effect. The labels used in such monitoring are preferentially those detectable by positron emission tomography (PET).

Thus, the present invention also offers a method to mimic the pharmacokinetics of a conjugate of a drug with respect to its behavior in the context of an EPR effect in solid tumors. By providing a suitable imaging agent with a carrier similar in size and shape to a carrier conjugated to a drug, the pharmacokinetics of the drug can be predicted by monitoring the pharmacokinetics of the imaging agent. Such diagnostic agents are also useful in the determining the suitability of treating patients with conjugates of therapeutic agents.

Thus, in one aspect, the invention is directed to an imaging agent of the formula (1)

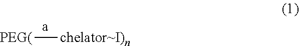

wherein PEG represents a polyethylene glycol comprising a plurality of 2-6 arms of 40-60 kD;
chelator represents a desferrioxamine or a plur-hydroxypyridinone multidentate;
I is a radioisotope suitable for positron emission tomography (PET);

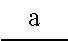

is a covalent connector;
˜ indicates sequestration of I in the chelator; and
n is an integer of 1 up to the number of arms of said PEG.

The invention also includes hybrid conjugates of formula (2)

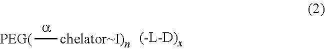

wherein PEG represents a polyethylene glycol comprising a plurality of 2-6 arms of 40-60 kD;
chelator represents a desferrioxamine or a plur-hydroxypyridinone multidentate;
I is a radioisotope suitable for positron emission tomography (PET);

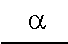

is a covalent connector;
˜ indicates sequestration of I in the chelator;
L is a linker;
D is a therapeutic agent;
n is an integer of 1 up to the number of arms of said PEG minus x, and
x is an integer of up to the number of arms of said PEG minus n.

The use of a multi-armed PEG is advantageous in that the resulting nanoparticle is less flexible, and thus retained more preferentially in tumors. The imaging agent will optimally have a diameter of approximately 20 nm (a hydrodynamic radius of approximately 10 nm). The diameter can be in the range of 10-100 nm, or 10-50 nm or 10-25 nm, corresponding to hydrodynamic radii of 5-50, 5-25 or 5-12.5 nm.

In another aspect, the invention is directed to a method to monitor accumulation of the imaging agent in a tumor which method comprises administering said imaging agent and detecting the location of said imaging agent by PET.

In still another aspect, the invention is directed to a method to assess the pharmacokinetics of a drug conjugate and its accumulation in tumor which method comprises matching the size of a conjugate of said drug to the size of the imaging agent, administering said imaging agent and monitoring the accumulation of said agent in the tumor by PET as diagnostic of the behavior of the drug conjugate.

Thus, the invention further includes method to assess suitability of treating a patient with a conjugated drug based on the diagnostic agent. The dimensions of the diagnostic agent are matched to those of a drug conjugate intended for patient treatment. More broadly the diagnostic agent can simply identify patients that can be treated taking advantage of the EPR effect.

The invention also includes kits that include the imaging agent of the invention and a conjugate of a drug of similar size and shape as the imaging agent.

In another aspect, the invention is directed to a method to identify a subject that will likely benefit from treatment with a drug modified to exhibit the EPR effect, which comprises administering the imaging agent of the invention to a candidate subject and monitoring the distribution of the imaging agent in the subject, whereby a subject that accumulates said imaging agent in an undesirable tissue mass is identified as a subject that will benefit from such treatment. See, for example, Lee, H., et al., Clin. Canc. Res., (2017) 23:4190-4202 (supra).

In connection with the protocols for treatment, the imaging agents of the invention having carriers with the same characteristics as those used in conjugating the drugs are used to monitor the uptake of the conjugates by the solid tumor. This permits verification (or not) that the corresponding conjugates of drugs will exhibit an EPR effect.

In a further aspect the invention includes a method to identify a subject having a tumor that will respond to treatment with an inhibitor of DNA repair which method comprises determining the presence or absence of a mutation in a gene that encodes a protein that participates in effecting DNA repair, wherein the presence of said mutation in the subject identifies the subject as having such a tumor.

In still another aspect the invention is directed to a hybrid conjugate for treatment and imaging of solid tumors which conjugate comprises a flexible carrier wherein the carrier is a nanoparticle or macromolecule each with a hydrodynamic radius of 5-50 nm which conjugate exhibits enhanced permeability and retention (EPR) in solid tumors so as to concentrate said conjugate in the tumor and wherein said carrier is releasably coupled to a therapeutic agent and also to an imaging agent, and to a method to correlate imaging and treatment of a solid tumor using said hybrid conjugate. An exemplary generic structure of such hybrids for any drug such hybrids designated as “theranostics” is shown in FIG. 12.

### BRIEF DESCRIPTION OF THE DRAWINGS

FIG. 1 is a graph showing the concentration of coupled SN-38 in the form of a conjugate to a four-armed 40 kD PEG (PLX038) in the plasma as a function of time. Similar results for the released SN-38 and the detoxified form of the drug, i.e., the glucuronide (SN-38G) are shown in the same figure. The rates are similar showing half-lives of 50 hours in the rat.

FIG. 2 shows the effect of various concentrations of PLX038 administered to the HT29 xenograft-bearing rat as compared to irinotecan.

FIGS. 3A and 3B show the concentrations of PLX038 in free SN-38 at various dosages in the tumor as compared to plasma.

FIG. 4 is a diagram showing a hypothetical dosing schedule in humans of a combination of PLX038 and a second drug (e.g., a poly ADP ribose polymerase (PARP) inhibitor) administered systemically. PLX038 is administered on day 1; the conjugate accumulates in the tumor and releases the free drug (dotted line) in the vicinity of the tumor and both conjugate and free drug are cleared from the system (solid line). After 2 half-lives of systemic clearance, in this case 10 days, systemic PLX038 is reduced to ˜25% of its original concentration, and the concentration lies below its minimal effective (and toxic) level. At this time the second drug is administered on an effective schedule.

FIG. 5 shows C vs. t plots of SN-38 released from PLX038 in the rat and from PLX038A in mouse. The curve for SN-38 released from PLX038 at 3.2 μmol (200 mg)/kg in the rat was modeled using previously determined pharmacokinetic parameters (Santi, D. V., et al., Proc. Natl. Acad. Sci. USA (2012) 109:6211-6216).

FIGS. 6A-6E are maximum intensity projections (MIP) at 72h and 120h of PEG40 kDa-DFB-⁸⁹Zr in mice bearing HT-29 xenografts (A) on both flanks overlaid on a CT scan; ex vivo biodistribution study of PEG40 kDa-DFB-⁸⁹Zr in mice bearing HT-29 xenografts (B) and tumor to blood ratios (C) vs time in mice bearing HT-29 tumors; 72h MIP image (D) of PEG-(SN-38)3-DFB-⁸⁹Zr in single flank tumor bearing mice and biodistribution of PEG-(SN-38)3-DFB-⁸⁹Zr (black) vs PEG-DFB-⁸⁹Zr (grey) at 72h (E).

FIGS. 7A-7C show the biodistribution of PEG_{40 kDa}-(DFB)-⁸⁹Zr₄ in mice bearing tumors.

FIG. 8 shows the biodistribution of various ⁸⁹Zr conjugates in HT-29 xenografts.

FIG. 9 shows the biodistribution of various ⁸⁹Zr conjugates in MX-J xenografts.

FIG. 10 shows the effectiveness of PEG-SN38 in tumor treatment.

FIGS. 11A-11C show synergy of an SN38 conjugate and separately administered talazoparib.

FIG. 12 shows a generic hybrid drug/label conjugate theranostic.

### MODES OF CARRYING OUT THE INVENTION

Essentially, there are two approaches to the design of protocols that minimize the toxic effects of combination therapies. The first approach is to ensure that a first therapeutic agent or drug is captured in a solid tumor to be treated by coupling the drug to a carrier such that the EPR effect results in substantially retaining the conjugate and released drug in the solid tumor, while the administered conjugate and released drug not captured in the tumor are more rapidly cleared from the systemic circulation, wherein the carrier is a nanoparticle or macromolecule each with a hydrodynamic radius of 5-50 nm preferably about 10 nm (diameter of 10-100 nm preferably about 20 nm). Thus a substantial portion of the administered conjugate is retained in the tumor, as well as is the drug that has been released from the conjugate while the conjugate resides in the tumor. As the clearance rate from the systemic circulation is much greater than the clearance rate of the conjugate and released drug from the tumor, an effective amount of drug both in conjugated and free form remain to exert a cytotoxic effect on tumor cells while their concentration in the systemic circulation has diminished to a desired level. After two half-lives in the systemic circulation, for example, the level of the conjugate and free drug in circulation and in contact with normal tissue is reduced to 25% of the initial concentration, and this may be sufficiently low to ameliorate toxicity. Since the conjugate remains in the tumor to release the agent, the agent is able to exert its cytotoxic effect on the tumor although its concentration in the systemic domain is quite low, and exposure of normal tissue to the drug is therefore also quite low.

At this point, a second drug is administered systemically and thus the normal tissue is exposed only to the toxic effect of the second drug while the first drug remains out of reach in the tumor. This minimizes the toxic effect of the combination on normal tissue while retaining the combined toxicities in the tumor. The second drug may be administered either in free form or it, too, may be administered as a conjugate with a similar carrier or in any other suitable form, including inclusion in delivery vehicles such as liposomes, nanoparticles, micelles, and the like.

In addition, a third drug that has non-overlapping toxicity with the second drug may be coadministered simultaneously or sequentially with said second drug.

Alternatively, both the first and second drug may be administered in the form of conjugates that are retained in the tumor by virtue of EPR either at the same time or at disparate times. By virtue of this retention, the major concentration of each drug occurs in the tumor rather than being in contact with normal tissue. Thus, the higher dosage levels of these drugs is experienced mainly in the tumor, and the administered conjugates along with released drug are rapidly cleared from the systemic circulation.

In some instances, still an additional conjugated form of an agent may be coadministered.

The carriers used in the method of the invention to administer at least the first agent in the first above-cited method and to release both the first and second agents in the second-noted method are carriers that are flexible in nature and have hydrodynamic radii of about 10 nm. Suitable macromolecule carriers include polyethylene glycols (PEG) which may be linear or multi-armed and have molecular weights of 10-50 kD. Preferably, the carriers are multi-armed PEG with molecular weights of at least 20 kD. These characteristics of the carriers assure that maximum advantage can be taken of the EPR effect. Nanoparticulate carriers are also included.

Particularly useful to provide a releasable form of a conjugate of the chemotherapeutic agents to nanomolecular carriers are linkers that release the agent by beta elimination reactions such as those described in detail in the above cited U.S. Pat. Nos. 8,680,315; 9,387,254; 8,754,190; 8,946,405; and 8,703,907 all incorporated herein by reference for their disclosures of not only the structure of useful linkers that release the agent by beta elimination, but also with respect to their disclosure of nanomolecular carriers useful in the present invention as well.

Other linkers include those cleavable by hydrolysis of esters, carbonates, or carbamates, by proteolysis of amides or by reduction of aromatic nitro groups by nitroreductase.

The subjects of the methods of the invention are typically human subjects, but the invention methods are also applicable in veterinary contexts including livestock and companion animals. The methods are also suitable for animal models useful in the laboratory such as rats, mice, rabbits or other model systems preparatory to designing protocols for human use.

With respect to the drugs useable in the combination therapy, a wide variety of chemotherapeutic agents is known and any combination of these may be selected as the first and second drug. Agents that act additively or synergistically are preferred, for example combination of drugs wherein each inhibits DNA repair.

Drugs that cause DNA damage, such as Topo 1 inhibitors, are particularly useful in treating tumors whose genome contains a mutation in a gene that normally aids in DNA repair. Among others, these genes include BRCA1, BRCA2, ATM which encodes ataxia telangiectasia mutated (ATM) kinase and ATR which encodes Rad-3 related (ATR) kinase. The invention includes identifying tumors that will show enhanced sensitivity to treatment with a Topo 1 inhibitor where the tumor-bearing subject's genome has at least one gene that has a mutation in BRCA1, BRCA2, ATM or ATR or other genes where mutation prevents or depresses the ability of the gene to enhance DNA repair. The response may be further enhanced by inhibiting a second enzyme involved in DNA repair, such as a PARP inhibitor, which then causes a synthetic lethality that is amplified because of the high level of DNA breaks caused by the Topo inhibitor. Thus, in using passively targeted PEG_SN38, it is useful to know the genetic status of the tumor, and to have an assortment choice of inhibitors of the DNA damage response system.

Examples of agents include:
“Signal transduction inhibitors” which interfere with or prevent signals that cause cancer cells to grow or divide;
“Cytotoxic agents”;
“Cell cycle inhibitors” or “cell cycle control inhibitors”—these interfere with the progress of a cell through its normal cell cycle, the life span of a cell, from the mitosis that gives it origin to the events following mitosis that divides it into daughter cells;
“Checkpoint inhibitors”—these interfere with the normal function of cell cycle checkpoints, e.g., the S/G2 checkpoint, G2/M checkpoint and G1/S checkpoint;
“Topoisomerase Inhibitors”, such as camptothecins, which interfere with topoisomerase I or II activity, enzymes necessary for DNA replication and transcription;
“Receptor tyrosine kinase inhibitors”—these interfere with the activity of growth factor receptors that possess tyrosine kinase activity;
“Apoptosis inducing agents”—these promote programmed cell death;
“Antimetabolites,” such as gemcitabine or hydroxyurea, which closely resemble an essential metabolite and therefore interfere with physiological reactions involving it;
“Telomerase inhibitors”—these interfere with the activity of a telomerase, an enzyme that extends telomere length and extends the lifetime of the cell and its replicative capacity;
“Cyclin-dependent kinase inhibitors”—these interfere with cyclin-dependent kinases that control the major steps between different phases of the cell cycle through phosphorylation of cell proteins such as histones, cytoskeletal proteins, transcription factors, tumor suppresser genes and the like;
“DNA damaging agents”;
“DNA repair inhibitors”;
“Anti-angiogenic agents”, which interfere with the generation of new blood vessels or growth of existing blood vessels that occurs during tumor growth; and
“Mitochondrial poisons” which directly or indirectly disrupt mitochondrial respiratory chain function.

Many combinations of these for treatment of tumors are the clinically approved.

Preferred agents that may be used in combination include DNA damaging agents such as carboplatin, cisplatin, cyclophosphamide, doxorubicin, daunorubicin, epirubicin, mitomycin C, mitoxantrone; DNA repair inhibitors including 5-fluorouracil (5-FU) or FUDR, gemcitabine and methotrexate; topoisomerase I inhibitors such as camptothecin, irinotecan and topotecan; S/G2 or G2/M checkpoint inhibitors such as bleomycin, docetaxel, doxorubicin, etoposide, paclitaxel, vinblastine, vincristine, vindesine and vinorelbine; G1/early S checkpoint inhibitors; G2/M checkpoint inhibitors; receptor tyrosine kinase inhibitors such as genistein, trastuzumab, ZD1839; cytotoxic agents; apoptosis-inducing agents and cell cycle control inhibitors.

Exemplary combinations are DNA damaging agents in combination with DNA repair inhibitors, DNA damaging agents in combination with topoisomerase I or topoisomerase II inhibitors, topoisomerase I inhibitors in combination with S/G2 or G2/M checkpoint inhibitors, G1/S checkpoint inhibitors or CDK inhibitors in combination with G2/M checkpoint inhibitors, receptor tyrosine kinase inhibitors in combination with cytotoxic agents, apoptosis-inducing agents in combination with cytotoxic agents, apoptosis-inducing agents in combination with cell-cycle control inhibitors, G1/S or G2/M checkpoint inhibitors in combination with cytotoxic agents, topoisomerase I or II inhibitors in combination with DNA repair inhibitors, topoisomerase I or II inhibitors or telomerase inhibitors in combination with cell cycle control inhibitors, topoisomerase I inhibitors in combination with topoisomerase II inhibitors, and two cytotoxic agents in combination.

Exemplary specific agents include cisplatin (or carboplatin) and 5-FU (or FUDR), cisplatin (or carboplatin) and irinotecan, irinotecan and 5-FU (or FUDR), vinorelbine and cisplatin (or carboplatin), methotrexate and 5-FU (or FUDR), idarubicin and AraC, cisplatin (or carboplatin) and taxol, cisplatin (or carboplatin) and etoposide, cisplatin (or carboplatin) and topotecan, cisplatin (or carboplatin) and daunorubicin, cisplatin (or carboplatin) and doxorubicin, cisplatin (or carboplatin) and gemcitabine, oxaliplatin and 5-FU (or FUDR), gemcitabine and 5-FU (or FUDR), adriamycin and vinorelbine, taxol and doxorubicin, flavopiridol and doxorubicin, UCN-01 and doxorubicin, bleomycin and trichlorperazine, vinorelbine and edelfosine, vinorelbine and sphingosine (and sphingosine analogues), vinorelbine and phosphatidylserine, vinorelbine and camptothecin, cisplatin (or carboplatin) and sphingosine (and sphingosine analogues), sphingosine (and sphingosine analogues) and daunorubicin and sphingosine (and sphingosine analogues) and doxorubicin.

In one embodiment, for a first drug is a releasable conjugate of the invention of SN-38, a topoisomerase inhibitor, exemplary second drugs include PARP inhibitors, mTOR inhibitors, trabectedin, cis-platinum, oxaliplatin, fluorouracil, temozolomide and vincristine—all of which have been reported to be synergistic with SN-38.

Certain tumors are especially susceptible to treatment with PARP inhibitors and in these tumors, PARP inhibitors are favored as the combination drug. These are tumors wherein a mutation in a gene that normally is helpful in providing a protein that aids in DNA repair takes away this property of the gene. Such tumors are also responsive to topoisomerase inhibitors, such as SN38, since inhibition of topoisomerase causes excess DNA damage that requires DNA repair that is deficient in these tumors. These genes include BRCA1, BRCA2, ATM which encodes ataxia telangiectasia mutated (ATM) kinase and ATR which encodes Rad-3 related (ATR) kinase, among others. The invention includes identifying tumors that have mutations in BRCA1, BRCA2, ATM or ATR or other genes where mutations prevent or depress the ability of the gene to enhance DNA repair and combining treatment with the invention SN38 conjugates with follow up treatment with for example PARP inhibitors, or other inhibitors of DNA repair. Because the drug accumulates and remains in the tumor after it is eliminated from the rest of the system, the toxicity of the SN38 is confined to the tumor and the system as a whole has only to deal with toxicity of the PARP inhibitor.

Some of the above listed drugs to be administered as second drugs may be administered in combination either sequentially or simultaneously provided their toxicities do not overlap.

### Imaging

Since the invention methods rely on the ability of the conjugates administered for the first agent in the first approach above and both the first and second agents in the second approach being subject to the EPR effect, it is important to confirm that this is in fact the case since tumors are heterogeneous and the particular carrier selected must be compatible with the pore structure of the vasculature in the solid tumor that resides in the subject in the sense that the EPR effect is present. Therefore, in some embodiments of the invention method, this is confirmed by administration either at the same time or separately of a conjugate of a label that is coupled non-releasably to the same carrier or a carrier with the same characteristics as that linked to the drug(s). While any detectable label, e.g., fluorescent label, can be used, it is most convenient to employ an isotope that is detectable by positron emission tomography (PET) scanning. The non-releasable conjugate of the isotope is then monitored to detect whether preferential uptake and retention by the tumor is exhibited. If so, the method of the invention is employed. If the tumor fails to exhibit the EPR effect with the labeled non-releasable conjugate, the method of the invention is contraindicated. The isotopes thus detectable are well known in the art as are means for coupling such isotopes to macromolecular carriers.

For imaging, a similar conjugate is used. As noted above, it is advantageous to design the imaging agent of the invention such that the diameter is approximately are 20 nanometers and to avoid excessive flexibility. This can be accomplished by using the multi-armed PEG polymers in the range of 40-60 kD. Although the number of arms associated with this polymer may range from 1-6, multi-armed PEGs of 3-5 arms, more preferably 4 arms are focused on herein.

The value of n in formula (1) can vary from 1 to the number of arms associated with the polymer and it should be understood that in the compositions of the invention the value of n may not be the same for all of the individual imaging moieties in the composition. Thus, for example, for a 4 armed PEG where n is 4, or in single chain PEG where n is 1, most of the individual “molecules” in a given composition will contain 4 or 1 as values of n respectively. However, for example for 4 armed PEG, for n=3 or n=2, represents an average and some of the individual entities may comprise 4, some comprise 3, some comprise 2 and some comprise 1 instances of n value

Further as to the structure of the imaging agent of Formula (1) noted above, the chelator represents a desferrioxamine or a multidentate chelator comprised of a multiplicity of hydroxypyridinones, abbreviated herein “plur-hydroxypyridinone multidentates.” A variety of such chelators are well known in the art and are described in detail, for example, in Ma, M. T. et al., Dalton Trans (2015) 44:4884-4900 and by Deri, M. A., J Med Chem (2014) 57:4849-4860. The description of these ligands in these documents is specifically incorporated herein by reference.

The covalent connector on Formula (1) may be a direct bond to the chelator or there may be intermediate linkers such as dipeptides or bifunctional linkers comprising 1-20 linking atoms. Radioisotopes (I) useful in PET in the context of the present invention are known in the art, and particularly a subset preferred among those set forth in Table 3 of Smith, S. V. et al., “Production and Selection of Metal PET Radioisotopes for Molecular Imaging,” in Radioisotopes—Applications in Bio-Medical Science, Nirmal Singh, ed., Chapter 10, InTech (Rijeka, Croatia), 2011, are those with suitable half-lives such as ⁸⁹Zr, ⁹⁴Tc, ¹⁰¹In, ⁸¹Rb, ⁶⁶Ga, ⁶⁴Cu, ⁶²Zn, ⁶¹Cu or ⁵²Fe.

To use the imaging agents of the invention as surrogates for delivery of active agents, i.e. drugs, the imaging agents contain carriers with the same characteristics as those carriers used in conjugating the drugs. These are then used to monitor the uptake of the conjugates by the solid tumor. This permits verification (or not) that the corresponding conjugates of drugs will exhibit an EPR effect.

An alternative to using separate therapeutic and imaging conjugates employs a hybrid conjugate of formula (2) for treatment and imaging of solid tumors which conjugate comprises a flexible carrier wherein the carrier is a nanoparticle or macromolecule each with a hydrodynamic radius of 5-50 nm which conjugate exhibits enhanced permeability and retention (EPR) in solid tumors so as to concentrate said conjugate in the tumor and wherein said carrier is releasably coupled to a therapeutic agent and also coupled to an imaging agent. Thus, in formula (2) as in formula (1),

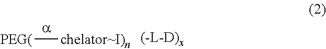

in some embodiments I is ⁸⁹Zr, ⁹⁴Tc, ¹⁰¹In, ⁸¹Rb, ⁶⁶Ga, ⁶⁴Cu, ⁶²Zn, ⁶¹Cu or ⁵²Fe, and/or the PEG is a four armed polyethylene glycol of approximately 40 kD, and n is 1-4, and/or the chelator is desferrioxamine-B, and/or

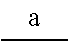

is a direct bond linkage.

As shown, at least one of the arms of the PEG is occupied by the imaging agent and at least one is occupied by the therapeutic agent. Various combinations up to the total number of arms of the PEG polymer are contemplated. The therapeutic agent may be SN38 or other topoisomerase inhibitor or any other suitable agent for tumor treatment that is benefited by accumulation in the tumor, such as a PARP or kinase inhibitor.

The imaging agents of the invention are also useful to identify subjects that harbor tumors or other tissue masses that are susceptible to treatment with therapeutic agents that exhibit the EPR effect. Thus, the imaging agent may be administered to a subject and monitored to determine whether the tumor, for example, will, in fact, preferentially take up and retain entities of similar size.

In this application, “a”, “an”, and the like are intended to mean one or more than one unless it is clear from the context that some other meaning is intended. In addition, the terms “chemotherapeutic agent”, “agent”, and “drug” are used interchangeably. Where specific numerical characteristics are set forth, the number cited will typically have error bars of plus-or-minus 10%, preferably plus-or-minus 5% and more preferably plus-or-minus 1%. Thus, a range of 10-50 nm could include a range of 9-55 nm. “Hydrodynamic radius” means the apparent Stokes radius— the radius of a hard sphere that diffuses through solution at the same rate as the molecule in question as measured, for example, by gel permeation chromatography.

The subjects of the invention are typically human, but also include non-human animals such as laboratory models and veterinary subjects.

All documents cited herein are hereby incorporated herein by reference.

The following examples are offered to illustrate but not to limit the invention.

### Example 1

### Administration of Conjugated SN-38

SN-38 is the topoisomerase I inhibitor that is the active drug released from the prodrug, irinotecan. Conjugates of SN-38 are described in WO 2015/051307. Two different conjugates of SN-38 were prepared: PLX038 and PLX038A. These conjugates couple the drug releasably to a four-armed PEG of 40 kD through a linker that effects release by O-elimination. The structure of PLX038 and PLX038A is shown below wherein “Mod” is —CN in PLX038, and methyl sulfonyl in PLX038A.

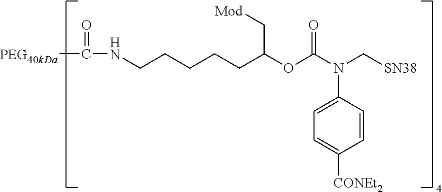

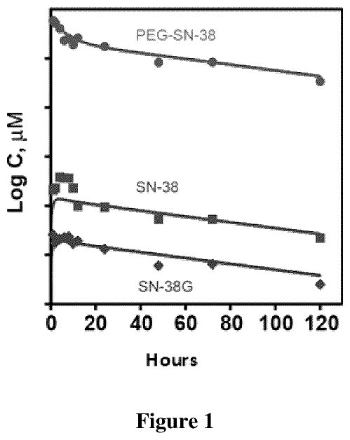

Six rats bearing HT29 tumor xenografts were injected with ˜200 mg/kg of PLX038 and the concentration in plasma and tumor of the conjugate and released drug as well as its glucuronide (SN-38G) were followed by HPLC with fluorescence monitoring As shown in FIG. 1, the half-life of PLX038 in the systemic circulation is about 50 hours. The conjugate and the free drug as well as SN-38G show similar half-lives.

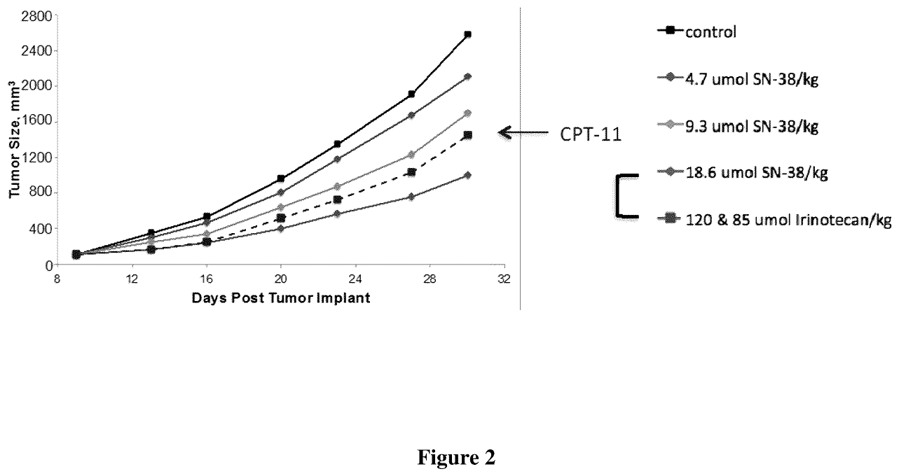

As shown in FIG. 2, the efficacy of a non-toxic dose of 20 nmol/kg of SN-38 in the form of PLX038 exceeds that of a toxic gastrointestinal dose of irinotecan control.

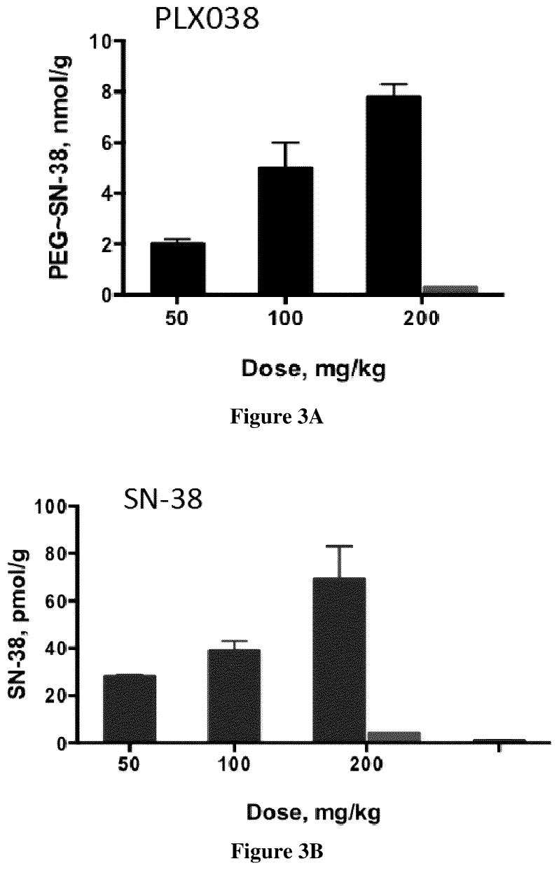

This is explained by the results shown in FIGS. 3A and 3B which are graphs of the levels of the conjugate PLX038 and of SN-38 that has been released from the conjugate in the tumor at various dosage levels. As seen in FIG. 3A, at an administered dose of 200 mg/kg the level of PLX038 in the tumor (a left bar) is roughly 8 nmol/g while the concentration in the plasma (shown as the right bar) is barely detectable. Similarly, in FIG. 3B with respect to the released SN-38, at the same dosage, the left bar shows the concentration in the tumor as about 80 pm/g while, again, the right bar shows that in the circulation it is barely detectable. Indeed, as shown, at the lower dosages, the conjugate and free drug are not detected in the plasma, while the tumor shows significant concentrations of these moieties.

### Example 2

### Suggested Human Protocol

A dosing schedule in humans for a combination of PLX038 and a second drug (e.g., a PARP inhibitor) administered systemically is proposed wherein PLX038 is administered on day 1 whereby the conjugate accumulates in the tumor and releases the free drug. The conjugate and the free drug are concomitantly cleared from the system. After two half-lives of systemic clearance or 10 days, systemic PLX038 is reduced to ˜25% of its original concentration, which lies below its minimal effective and toxic levels. At this time the second drug, which is synergistic with SN-38 is administered orally for 20 days.

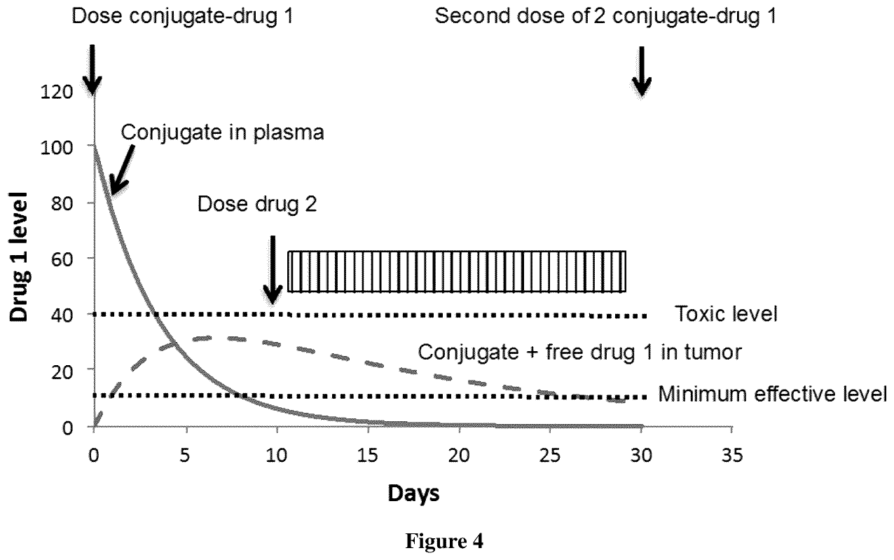

As shown in FIG. 4, the EPR effect concentrates PLX038 in the tumor (dotted line), while the systemic PLX038 (solid line) is sufficiently low that any toxic effect is only to a second drug, which is administered as shown at 10 days. At that time, the concentration of the conjugate in the tumor is still above the minimum effective level but below the toxic level.

### Example 3

### Design of a Mouse Model

Because most xenograft tumor models use mice as hosts, it is desirable to adapt the protocols of the present invention to testing in mice. Adaptation is needed because the half-life of the PLX038 conjugate in the mouse is only about 24 hours, whereas in the rat it is about 48 hours and in humans about 6 hours. Because the more rapid elimination of PLX038 in mice occurs before substantial amounts of the SN-38 are released, a different conjugate of SN-38, PLX038A that has a higher cleavage rate, is used in murine experiments.

Linker cleavage is species independent. While 32% of PLX038 is converted to SN-38 over one half-life of the conjugate in humans, only 12% is converted in the rat and 6% in the mouse. For PLX038A, the cleavage half-life is 70 hours and 26% conversion to SN-38 over one half-life of the conjugate in the mouse occurs. This conjugate also can be administered intraperitoneally (IP) in mice with 100% bioavailability.

However, in mice PLX038A still has a short t_{1/2} of renal elimination so a single dose may not effect high tumor accumulation and longer exposure may be necessary to achieve this. A multi-dose schedule for PLX038A in the mouse that simulates a single effective dose of the conjugate that gives high tumor accumulation in the rat is therefore used.

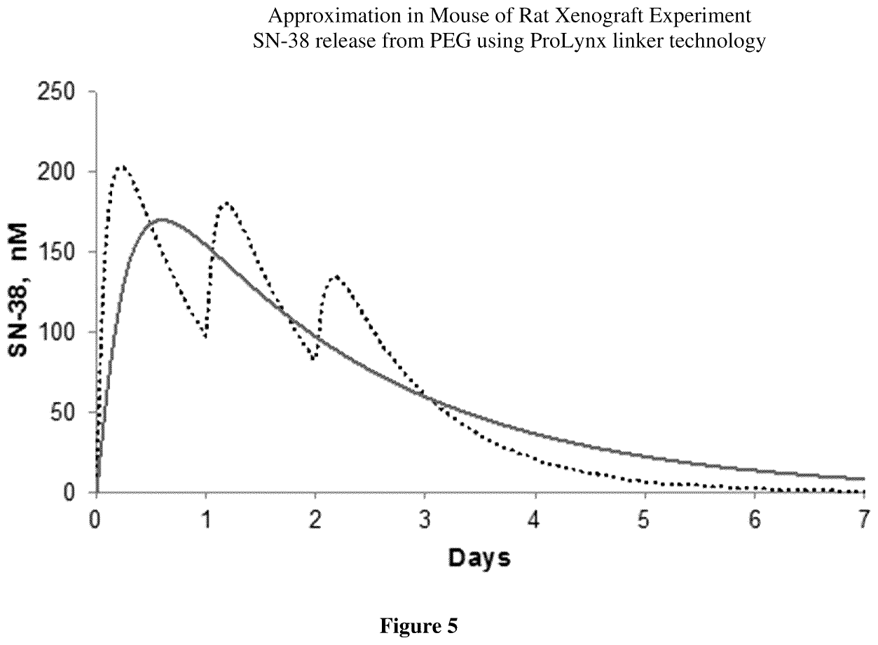

For comparison, in the rat xenograft model for colon cancer (HT-29), a single 200 mg/kg of PLX038 produced 61% inhibition of tumor growth with no gastrointestinal (GI) toxicity while irinotecan control that showed near-equal tumor inhibition showed significant GI toxicity. There was high accumulation of PLX038 and SN-38 in tumors 14 days post-dosing when the serum levels were undetectable. A dosing schedule for PLX038A in the mouse that would simulate the pharmacokinetics (PK) of PLX038 in the rat is shown in FIG. 5. Three daily decreasing doses of 152, 60 and 54 mmol/kg effectively simulate the rat PK profile of released SN-38 from PLX038. The “effective” half-life of SN-38 in the schedule is over 2 days, whereas SN-38 from irinotecan in the mouse is ˜2 hours. The data supporting FIG. 5 are shown in Table 1.

TABLE 1

| mouse dose | | conj dose, | SN-38 | SN-38 dose, | | |
|---|---|---|---|---|---|---|
| schedule | | mg | dose, mg | nmol | | |
| dose 1 | | 1.7 | 0.060 | 152 | | |
| dose 2 | | 0.9 | 0.032 | 80 | | |
| dose 3 | | 0.6 | 0.021 | 54 | | |
| total | | 3.2 | 0.1 | 285.4 | | |
| | | conj | SN-38 | SN-38 | | time |
| | | dose, | dose, | dose, | AUC, | over |
| | | mg/kg | mg/kg | μmol | μM-h | 8 nM |
| rat | dose | 200 | 7 | 3.2 | 11 | ~7 |
| | | | | | | days |
| mouse | total | 128 | 4.5 | 0.285 | 11 | ~5 |
| | dose | | | | | days |

### Example 4

### Murine Testing

The ability of HT29 xenografts to accumulate conjugate using the EPR effect is tested by injecting mice with one dose IP of 50 nmol of 40 kD four-armed PEG fluorescein per 100 g (15 nmol/mouse) to obtain about 10 M in serum. Blood and tumor are sampled at various times (at 6, 24, 48 and 96 hours) and the level of fluorescein measured. (The tumor is excised and digested with sodium hydroxide for measurement.)

PLX038A is tested for tumor growth inhibition in a nude mouse HT29 tumor xenograft using the three-dose schedule developed in Example 3.

The nude mouse model with HT29 xenograft is treated with the three-dose schedule of PLX038A developed in Example 3 and at 14 days the mice were treated daily with oral administration of a PARP inhibitor.

A conjugate of PARP inhibitor analogous to PLX038A is administered daily to nude mice bearing HT29 tumors and tested vs. daily administration of free inhibitor.

Combinations of conjugates PLX038A and the relevant conjugate of PARP inhibitor are also tested concomitantly in this model.

### Example 5

### Synthesis of PEG_{40 kDa}-PET Isotopes

### Synthesis of PEG-Desferrioxamine Conjugates

### 4-Branched PEG₄₀ kDa Coupled to DFB:

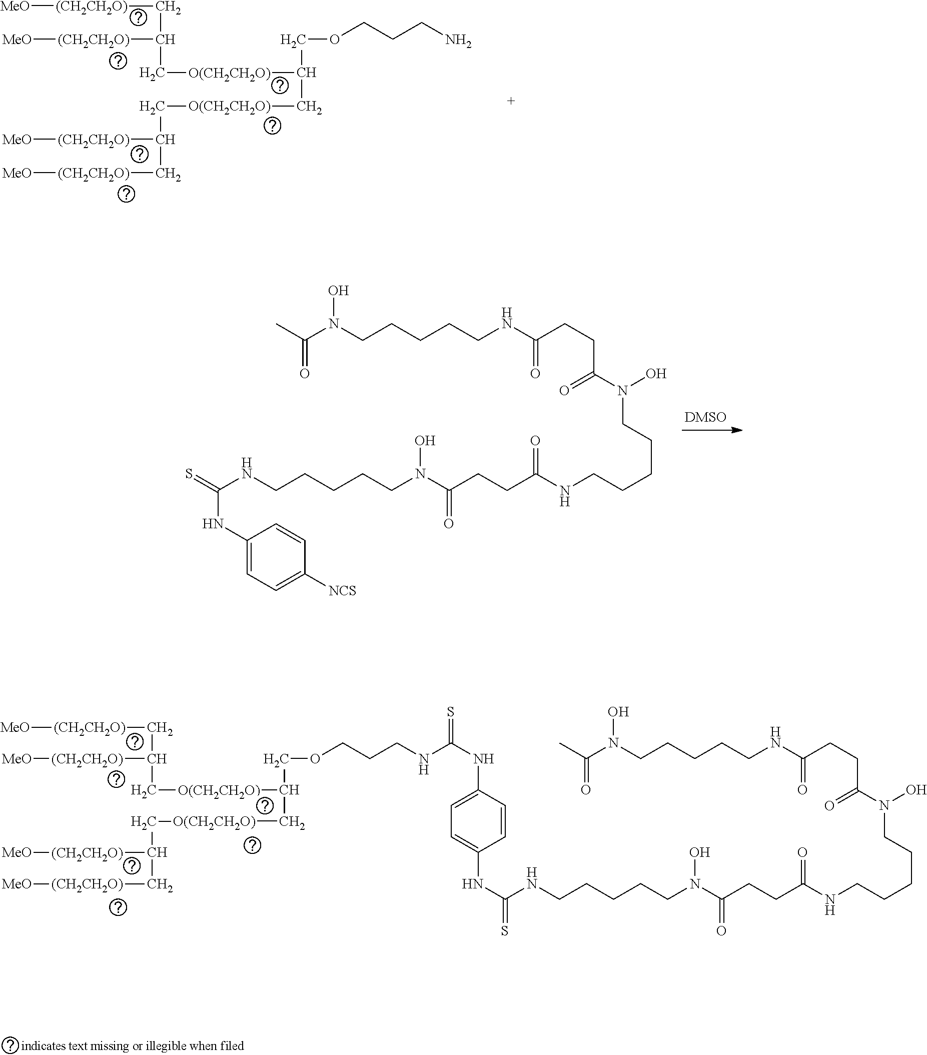

A solution of 4-branched 40-kDa PEG-amine (GL4-400PA, NOF; 150 mg, 3.75 μmol) and p-isothiocyanatobenzyl-desferrioxamine B (Macrocyclics; 4 mg, 5.3 μmol) in 2 mL of DMSO was kept 16 hat ambient temperature, then dialyzed against water (SpectraPor 2 membrane, 12-14 kDa cutoff) to remove unconjugated materials. The solution was evaporated to dryness, and the residue was dissolved in 2 mL of THF and added slowly with stirring to 50 mL of MTBE. The precipitated conjugate was collected and dried to provide the product (140 mg). A 2.4-mg sample was dissolved in 58 μL of water to give a 1 mM solution. A 20-uL aliquot was added to 100 μL of 1 mM Fe(III) perchlorate, giving a solution showing OD42snm=0.459. Based on an extinction coefficient of 2,300 M−1cm−1, this indicated a DFB concentration on 1.1 mM, in good agreement with expected.

(PEG)₄₀ coupled to [DFB=Desferrioxamine B](DFB): PEG₄₀ kDa-(DFB)₄ was prepared by reaction of PEG_{40 kDa}(NH₂)₄ with p-isothiocyanatobenzyl-DFB (Perk, L. R., et al. Eur. J. Nucl. Med. Mol. L (2010) 37:250-259; Fischer, G., et al., Molecules (2013) 18:6469-6490; and van de Watering, F. C., et al. Biomed. Res. Int. (2014) 2014:203601) (macrocyclics) as follows.

4-Armed PEG_{40 kDa} Coupled to DFB (PEG_{40 kDa}-(DFB)4]:

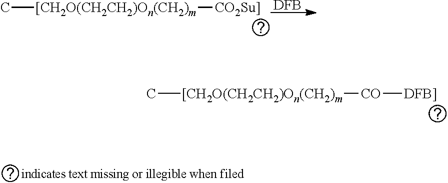

A solution of 40-kDa 4-armed PEG-tetra(succinimidyl ester) (JenKem Technologies; 100 mg, 10 μmol succinimidyl ester), deferoxamine mesylate (Sigma; 10 mg, 15 μmol), N,N-diisopropylethylamine (35 uL, 200 μmol), and HATU (1-[Bis(dimethylamino)methylene]-1H-1,2,3-triazolo [4,5-b]pyridinium 3-oxide hexafluorophosphate) (7 mg, 18 μmol) in 2 mL of DMF was kept 16 h at ambient temperature, then dialyzed against water and methanol (SpectraPor 2 membrane, 12-14 kDa cutoff) to remove unconjugated materials. The solution was evaporated to dryness, and the residue was dissolved in 2 mL of THF and added slowly with stirring to 50 mL of MTBE to give the conjugate (84 mg). A 5.0 mg aliquot was dissolved in 500 μL of water to give a solution 0.21 mM solution of conjugate. Assay for DFB content by addition to 1 mM Fe(III) perchlorate as described above gave 0.84 μM DFB, indicating 4 DFB per conjugate.

### Alternative Method

An alternative DFB reagent for conjugation, is prepared by acylation of DFB with N₃—(CH₂)ₙCO—HSE; the N₃—(CH₂)ₙCO-DFB is reacted with cyclooctyne-derivatized-PEG_{40 kDa}(NH₂)₄ by SPAAC.

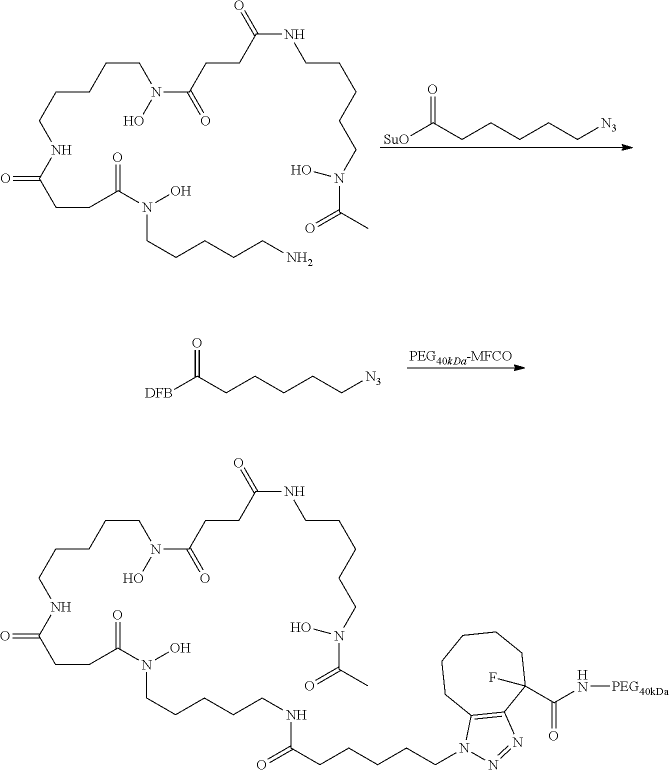

### Coupling to PET Isotopes:

Coupling to PET isotopes was performed by treatment of the PEGylated-DFB with ⁸⁹Zr oxalate followed by purification using size-exclusion chromatography (Perk, L. R., supra; and van de Watering, F. C., supra).

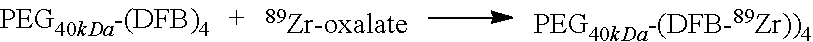

PEG_{40 kDa}-(BzI¹²⁵I)₄ is prepared by reacting the ¹²⁵I-azide shown below with a cyclooctyne-derivatized-PEG_{40 kDa}(NH₂)₄ (prepared from MFCO—HSE+PEG_{40 kDa}(NH₂)₄), which results in a clean high yield strain-promoted azide-alkyne cycloaddition (SPAAC) reaction. Preparation and radioiodination of the [¹²⁵I] iodobenzoyl-PEG-azide is shown below for stable iodination of macromolecules using SPAAC.

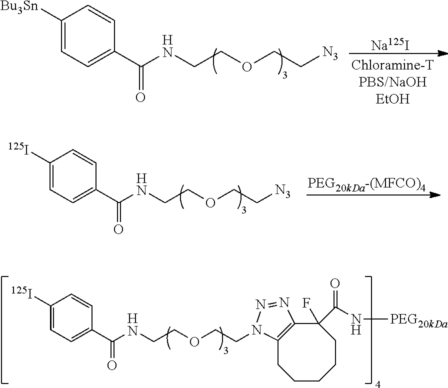

### Example 6

### Hybrid SN38/DFB Conjugates

4-armed PEG_{40 kDA} coupled to 1× stable-DFB and 3× releasable-SN-38 (PEG_{40 kDA}-(sDFB)₁(rSN38)₃):

### A. Preparation of Hybrid SN38/DFB Conjugates.

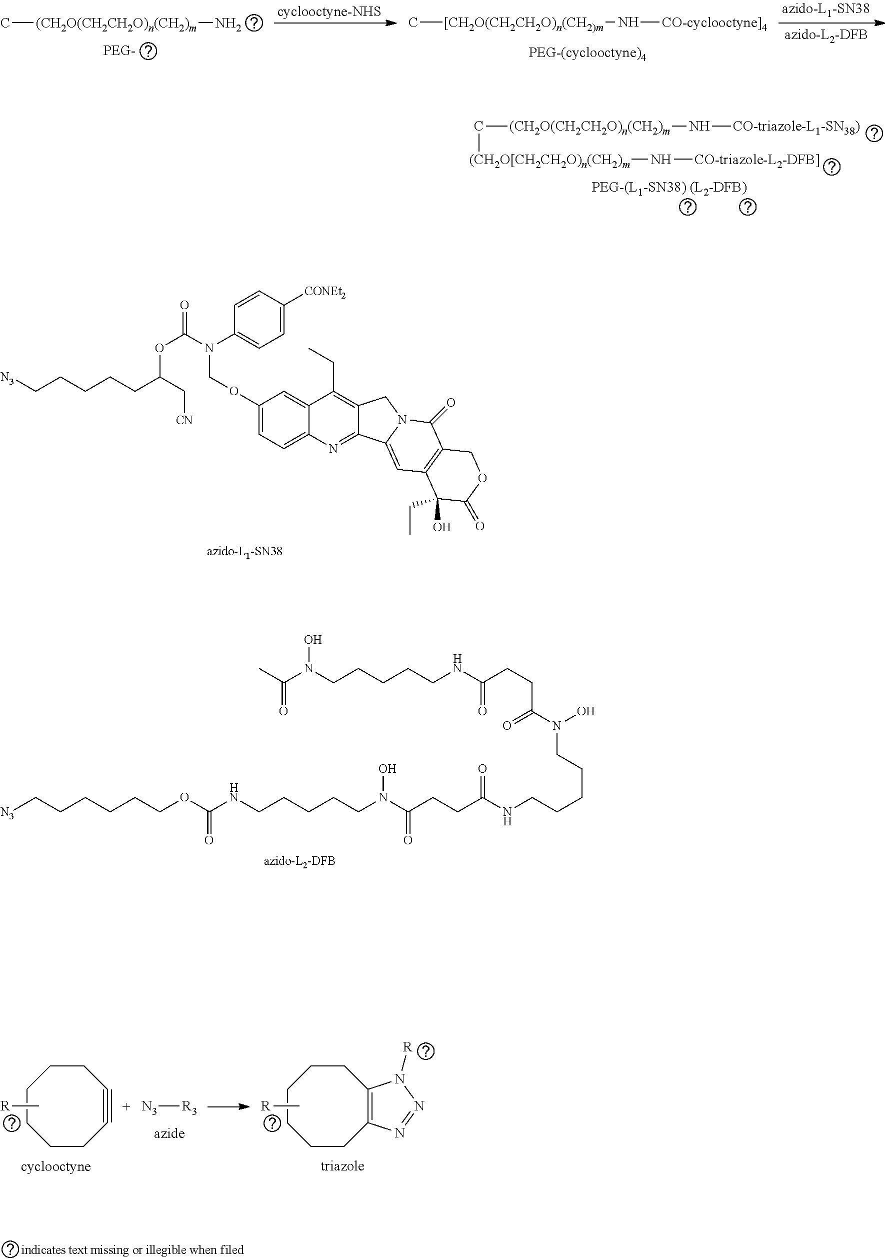

N-((6-azidohexyloxy)carbonyl) desferrioxamine B: A solution of 6-azidohexyl succinimidyl carbonate (35 mg, 120 μmol) in 2 mL of acetonitrile was added to a solution of deferoxamine mesylate (65 mg, 100 μmol) in 2 mL of 0.5 M NaHCO₃. After stirring for 16 h, the resulting white precipitate was collected, washed with water and acetonitrile, then dried under vacuum to yield the product (45 mg; 62%). MS: [M+H]⁺=730.46 (calc. for C₃₂H₆₀N₉O₁₀=730.44).

Azido-linker-SN38 having a cyano modulator: prepared as described in PCT Publication WO2015/051307.

PEG_{40 kDa}-(DBCO)₄: A solution of 40-kDa 4-armed PEG-tetraamine (PTE400-PA, NOF; 10 umol amines), dibenzocyclooctyne-N-hydroxysuccinimidyl ester (DBCO-NHS, ClickChemistryTools; 5 mg, 12 umol), and N,N-diisopropylethylamine (2 uL, 12 umol) in 1 mL of acetonitrile was stirred for 1 h at ambient temperature. The mixture was evaporated to dryness, then redissolved in 1 mL of THF and precipitated by addition of 10 mL of MTBE. The resulting solid was collected, washed with MTBE, and dried to provide the product.

PEG_{40 kDa}-(sDFB)₁(rSN38)₃: A 1:3 mixture of stable-linker-DFB and releasable-linker-SN38 was coupled to PEG_{40 kDa}(DBCO)4 to yield a mixture that was predominantly PEG_{40 kDa}(sDFB)₁(rSN38)₃ and PEG_{40 kDa}(rSN38)4 by HPLC analysis. These were separated by preparative HPLC using a Phenomenex 300A 5 um Jupiter C18 column, 21.2×150 mm, with a 30-60% gradient of acetonitrile in water+0.1% TFA at 15 mL/min. Determination of SN38 content by UV at 360 nm (e₃₆₀=22,400 M⁻¹cm⁻¹) and DFB content by assay with Fe(III) perchlorate as described above gave a 2.7:1 ratio of SN-38 to DFB.

### B. Preparation of Additional Hybrid Drug/DFB Conjugates.

i. Alternate Preparation of (5HCO)₃—PEG_{40 kDa}-DFB Intermediate

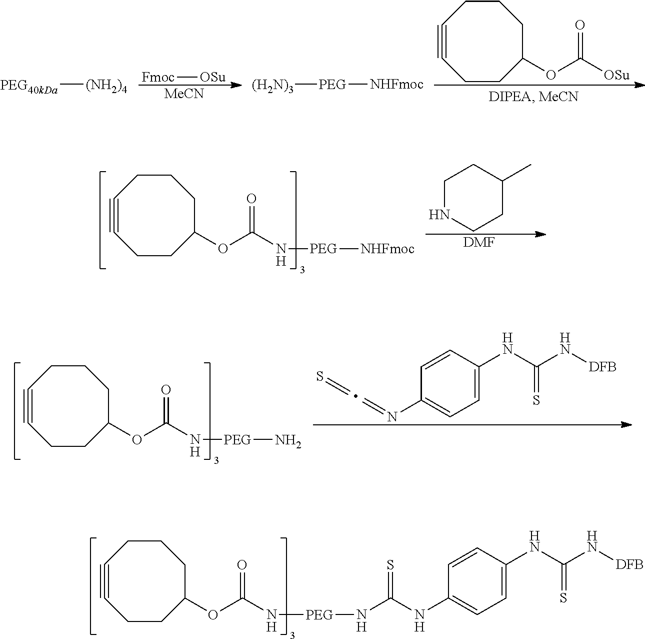

Step 1. (H₂N)₃—PEG_{40 kDa}-NHFmoc. A 25 mM solution of Fmoc-OSu (0.48 mL, 12 μmol) in MeCN was added dropwise to a vigorously stirred solution of PEG_{40 kDa}-(NH₂)4 (406 mg, 10.0 mol, 5 mM final concentration) in 3.5 mL of MeCN. The reaction mixture was stirred at ambient temperature, and after 5 min, the mixture consisted of 44% title compound as judged by C18 HPLC (ELSD). The reaction solution was concentrated to ˜1 mL by rotary evaporation. The concentrate was diluted to 6 mL with H₂O (0.1% TFA) then purified by preparative C18 HPLC, two injections eluting with a linear gradient (35%-60%) of MeCN in H₂O (0.1% TFA). Fractions from the first eluting Fmoc-containing peak were analyzed by C18 HPLC, and clean, product-containing fractions were combined and concentrated to dryness. After removing volatiles under high vacuum for 30 min, the residue was dissolved in minimal THF (˜1 mL) and added dropwise to 40 mL of 0° C. MTBE in a tared 50 mL Falcon tube. The suspension was vortexed, kept on ice for 15 min, centrifuged (3500× g, 1 min), and decanted. The precipitate was washed with MTBE (2×40 mL), isolated as above, and dried under high vacuum to provide the title compound (96 mg, 2.2 mol given 3 TFAs, 22% yield) as a white powder. C18 HPLC, purity was determined by ELSD: 99.6% (RV=9.39 mL).

Step 2. (Cyclooct-4-yn-1-yloxycarbonyl-NH)3-PEG_{40 kDa}-NHFmoc. A 0.15 M solution of O-(cyclooct-4-yn-1-yl)-O′-succinimidyl carbonate (63 μL, 9.5 μmol) in MeCN was added dropwise to a stirred solution of (H₂N)₃—PEG_{40 kDa}-NHFmoc (96 mg, 2.2 μmol, 50 mg/mL final concentration; 6.7 μmol NH₂) and DIPEA (2.8 μL, 16 μmol) in 1.9 mL of MeCN. The reaction mixture was stirred at ambient temperature and monitored by C18 HPLC. The starting material was converted to a single product peak via two slower eluting intermediate peaks. After 2 h, the reaction mixture was concentrated to ˜0.3 mL by rotary evaporation. The concentrate was diluted with 1 mL of THF, and the solution was added dropwise to 40 mL of ice-cold MTBE in a tared 50 mL Falcon tube. The mixture was kept on ice for 15 min then centrifuged (3500× g, 1 min) and decanted. The wet solid was washed with ice-cold MTBE (2×40 mL), centrifuged (3500× g, 1 min) and decanted. Residual volatiles were removed under high vacuum for 20 min to provide the title compound (40 mg, 0.93 μmol, 66% yield) as a white powder. To prevent decomposition, the solid was immediately diluted with 0.78 mL of amine-free DMF. C18 HPLC, purity was determined by ELSD: 93.5% (RV=9.96 mL) and a 6.5% impurity (RV=9.78 mL).

Step 3. (Cyclooct-4-yn-1-yloxycarbonyl-NH)₃—PEG_{40 kDa}-NH₂. 4-Methylpiperidine (39 μL, 5% v/v final concentration) was added to a 100 mg/mL solution of (cyclooct-4-yn-1-yloxycarbonyl-NH)₃—PEG_{40 kDa}-NHFmoc (0.78 mL, 78 mg, 1.8 mol) in DMF. The reaction tube was kept at ambient temperature and monitored by C18 HPLC. After 30 min, PEG was precipitated by dropwise addition of the reaction solution to 40 mL of ice-cold MTBE in a tared 50 mL Falcon tube. The mixture was kept on ice for 15 min then centrifuged (3500× g, 1 min) and decanted. The wet solid was washed with MTBE (2×40 mL), centrifuged (3500× g, 1 min) and decanted. Residual volatiles were removed under high vacuum for 15 min to provide the title compound (68 mg, 1.6 mol, 89% yield) as a white powder. To prevent decomposition, the solid was immediately diluted with 0.68 mL of amine-free DMF. C18 HPLC, purity was determined by ELSD: 87.0% (RV=9.59 mL) and a 13.0% impurity (RV=9.43 mL).

Step 4. (Cyclooct-4-yn-1-yloxycarbonyl-NH)₃—PEG_{40 kDa}-NHCSNH-phenyl-4-(NHCSNHDFB). P-isothiocyanatobenzyl-desferrioxamine B (1.8 mg, 2.4 mol; Macrocyclics) was added to a 50 mg/mL solution of (cyclooct-4-yn-1-yloxycarbonyl-NH)₃-PEG_{40 kDa}-NH₂ (1.36 mL, 1.6 mol) in DMF. The reaction mixture was placed in a 37° C. water bath and monitored by C18 HPLC. After 4 h, PEG was precipitated by dropwise addition of the reaction solution to 40 mL of ice-cold MTBE in a tared 50 mL Falcon tube. The mixture was kept on ice for 15 min then centrifuged (3500× g, 2 min) and decanted. The wet solid was washed with MTBE (2×40 mL), centrifuged (3500× g, 2 min) and decanted. Residual volatiles were removed under high vacuum for 15 min to provide the title compound (67 mg, 1.5 mol, 94% yield) as a white solid. To prevent decomposition, the solid was immediately diluted to 2.68 mL total volume with MeCN (2.61 mL MeCN, 25 mg/mL). Insoluble DFB-NCS was pelleted (3500× g, 2 min), and the product-containing MeCN supernatant was removed. C18 HPLC, purity was determined by ELSD: 80.3% (RV=9.59 mL) and a 19.7% shoulder (RV=9.43 mL).

ii. Preparation of (Drug)₃-PEG_{40 kDa}-DFB
a. Drug=SN38

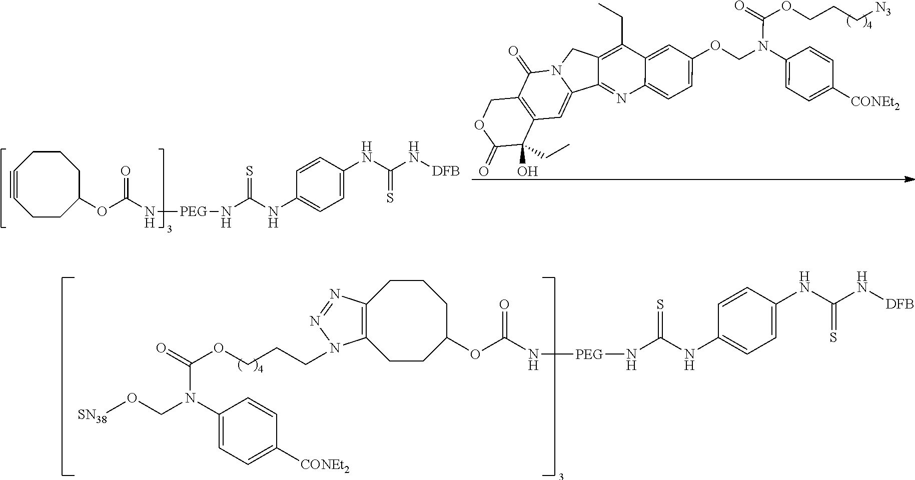

(SN38-L)₃-PEG_{40 kDa}-NHCSNH-phenyl-4-(NHCSNH-DFB). Stable azido-SN38 (4.0 mg 5.2 mol, 4 mM final concentration; Santi et al., J. Med. Chem. 57: 2303-14 (2014)) was added to a 25 mg/mL solution of (cyclooct-4-yn-1-yloxycarbonyl-NH)₃-PEG_{40 kDa}-NHCSNH-phenyl-4-(NHCSNHDFB) (1.3 mL, 0.75 mol PEG, 2.3 mol cyclooctyne, 1.8 mM cyclooctyne final concentration) in MeCN. The reaction was placed in a 37° C. water bath and monitored by C18 HPLC. After 44 h, the reaction solution was dialyzed against MeOH (12-14 k MWCO). The dialysate was concentrated to dryness, and residual volatiles were removed under high vacuum to provide the title compound (24 mg, 0.52 mol, 69% yield by mass) as white film that contained 1.4 mol of SN38 as determined by A₃₈₃ and 0.50 mol of DFB as determined by A₄₉₀ of Fe³⁺-DFB. The SN38:DFB ratio was found to be 2.8:1 using SN38 ε₃₈₃=29,100 M⁻¹ cm⁻¹ and Fe³⁺-DFB ε₄₉₀=3,000 M⁻¹ cm⁻¹. C18 HPLC, purity was determined by ELSD: 83.0% (RV=9.67 mL) and a 14.6% shoulder (RV=9.52 mL).

b. Drug=Rucaparib—A PARP Inhibitor

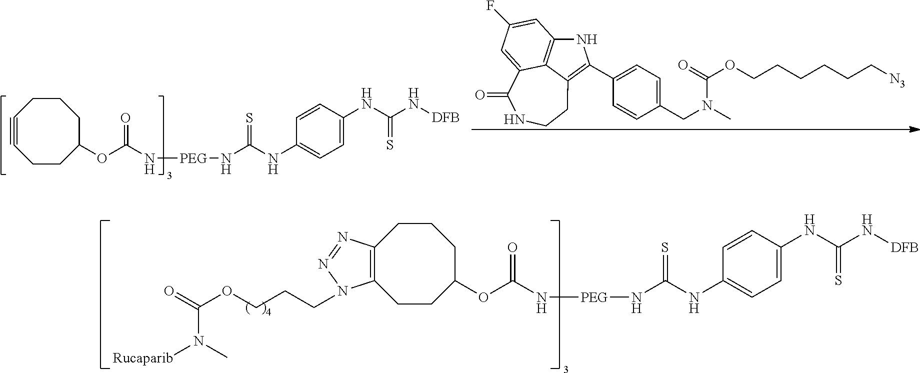

(Rucaparib-L)₃-PEG_{40 kDa}-NHCSNH-phenyl-4-(NHCSNH-DFB). A 10 mM solution of stable azido-rucaparib (0.11 mL, 1.1 mol, 1.8 mM final concentration; prepared by reacting rucaparib with 6-azidohexyl succinimidyl carbonate according to the procedures of Santi et al., Proc. Natl. Acad. Sci. 109: 6211-16 (2012)) was added to a 25 mg/mL solution of (cyclooct-4-yn-1-yloxycarbonyl-NH)₃—PEG_{40 kDa}-NHCSNH-phenyl-4-(NHCSNHDFB) (0.50 mL, 0.29 mol PEG, 0.86 mol cyclooctyne, 1.4 mM cyclooctyne final concentration) in MeCN. The reaction was placed in a 37° C. water bath and monitored by C18 HPLC. After 68 h, the reaction solution contained a ˜35:65 mixture of unmodified:PEGylated drug-linker. A series of the individual species of (drug)n-PEG-DFB was not observed. The reaction solution was concentrated by SpeedVac to 0.1 mL, diluted to 1.0 mL with H₂O, and loaded onto a PD-Midi column. Elution with H₂O yielded a cloudy fraction of excluded material that contained both unmodified and PEGylated drug-linker. The mixture was then dialyzed against MeOH (12-14 k MWCO). The dialysate was concentrated to dryness, and residual volatiles were removed under high vacuum to provide the title compound (8.7 mg, 0.19 mol, 66% yield) as white film that contained 0.51 mol of rucaparib as determined by A₃₅₅ and 0.19 mol of DFB as determined by A₄₉₀ of Fe³⁺-DFB. The rucaparib:DFB ratio was found to be 2.7:1 using rucaparib ε₃₅₅=13,260 M⁻¹ cm⁻¹ (125SF68) and Fe³⁺-DFB ε₄₉₀=3,000 M⁻¹cm⁻¹. C18 HPLC, purity was determined by ELSD: 78.5% (RV=9.41 mL) and a 21.5% shoulder (RV=9.27 mL).

c. Drug=VX-970—an ATR Kinase Inhibitor

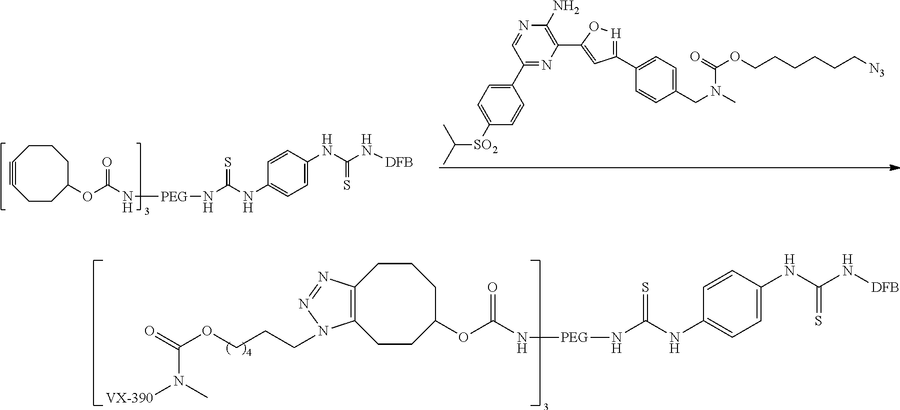

(VX970-L)₃-PEG_{40 kDa}-NHCSNH-phenyl-4-(NHCSNH-DFB). As described above for rucaparib, stable azido-VX970 (0.11 mL, 1.1 mol, 1.8 mM final concentration; prepared by reacting VX970 with 6-azidohexyl succinimidyl carbonate according to the procedures of Santi et al., Proc. Natl. Acad. Sci. 109: 6211-16 (2012)) was treated with a 25 mg/mL solution of (cyclooct-4-yn-1-yloxycarbonyl-NH)₃—PEG_{40 kDa}-NHCSNH-phenyl-4-(NHCSNHDFB) (0.50 mL, 0.29 mol PEG, 0.86 mol cyclooctyne, 1.4 mM cyclooctyne final concentration) in MeCN to provide the title compound (10 mg, 0.22 mol, 76% yield by mass) as white film that contained 0.55 mol of VX970 as determined by A₃₈₃ and 0.24 mol of DFB as determined by A₄₉₀ of Fe³⁺-DFB. The VX970:DFB ratio was found to be 2.3:1 using VX970 ε₃₈₃=17,200 M⁻¹ cm⁻¹ (127BH52) and Fe³⁺-DFB ε₄₉₀=3,000 M⁻¹ cm⁻¹. C18 HPLC, purity was determined by ELSD: 59.2% (RV=9.98 mL) and a 38.4% shoulder (RV=9.73 mL).

### D. Drug=BMN673—a PARP Inhibitor

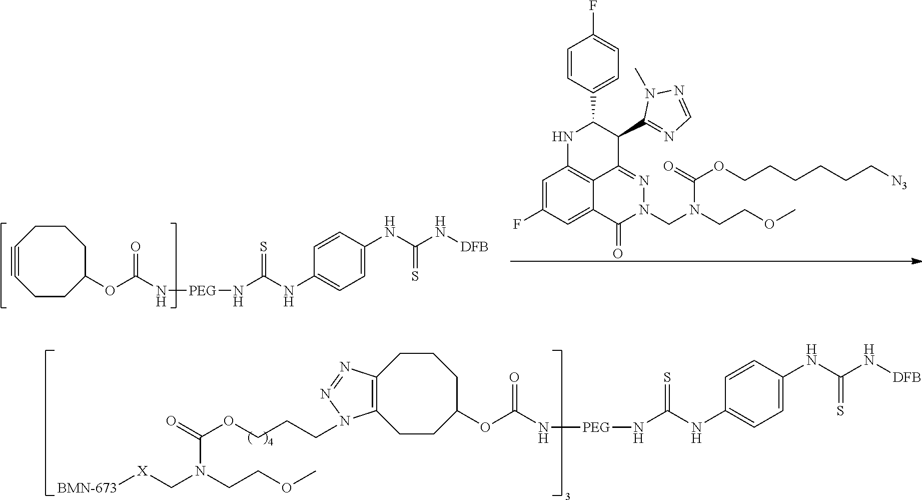

(BMN673-L)₃-PEG_{40 kDa}-NHCSNH-phenyl-4-(NHCSNH-DFB). As described above for rucaparib, stable azido-BMN673 (0.11 mL, 1.1 mol, 1.8 mM final concentration; prepared by reacting BMN673 with 6-azidohexyl succinimidyl carbonate according to the procedures of Santi et al., Proc. Natl. Acad. Sci. 109: 6211-16 (2012)) was treated with a 25 mg/mL solution of (cyclooct-4-yn-1-yloxycarbonyl-NH)₃—PEG_{40 kDa}-NHCSNH-phenyl-4-(NHCSNHDFB) (0.50 mL, 0.29 mol PEG, 0.86 mol cyclooctyne, 1.4 mM cyclooctyne final concentration) in MeCN to provide the title compound (12 mg, 0.26 mol, 91% yield by mass) as white film that contained 0.65 mol of BMN673 as determined by A₃₁₀ and 0.20 mol of DFB as determined by A₄₉₀ of Fe³⁺-DFB. The BMN673:DFB ratio was found to be 3.3:1 using BMN673 ε₃₁₀=9872 M⁻¹ cm⁻¹ (125SF39) and Fe³⁺-DFB ε₄₉₀=3,000 M⁻¹ cm⁻¹. C18 HPLC, purity was determined by ELSD: 69.7% (RV=9.47 mL) and a 30.3% shoulder (RV=9.32 mL).

### C. Coupling to PET Isotopes

The hybrid SN38/DFB and alternative hybrid drug/DFB conjugates are coupled to ⁸⁹Zr by the methods set forth in Example 5.

### Example 7

### Use of PET to Detect EPR in Animal Studies

Mice bearing HT-29 human xenografts and normal mice are treated with conjugates PEG-PET isotopes which are similar in size and shape to the drug conjugates of Examples 1-4. PET-imaging to measure accumulation of labeling intensity of the tumor at t=0, 12, 24, 48 and 96 hr is conducted in comparison with results of a similar experiment using PEG_{40 kDa}-fluorescein in tumor-bearing mice (Singh, Y., supra). Sera are counted at these time points to determine the t_{1/2} of elimination of the PEG-isotope (the elimination t_{1/2} of PEG_{40 kDa} in mice is usually ˜24 hr), as well as total body radioactivity measurements.

HT-29 Tumor bearing mice and normal control mice are treated with ˜200 uCi/mouse, and PET-imaging is performed at varying times to determine the amount and rates of accumulation. A signal is observable at ˜1 uCi/cc so the tumor is easily visualized as long as the background tissue does not accumulate the tracer. In the same experiment, the loss of isotope is followed as the reagent is cleared from the body. Rates of a) tumor accumulation of the PEG-isotope (quantitative PET imaging), b) vascular elimination (serum radioactivity), c) systemic elimination (whole body radioactivity) and d) tumor elimination (quantitative PET imaging) are thus determined.

At a time when tumor accumulation is complete, tumor-bearing mice are treated with varying amounts of the PEG_{40 kDa}-isotope to determine the maximal amount of nanoparticle that can accumulate.

Thus, in this example, PET scanning is used to simulate the behavior of an agent coupled to the same or similar carrier to evaluate the parameters appropriate for the drug administration protocol.

### Example 8

### PET Imaging/Biodistribution of Peg_{40 kDa}-Dfb⁸⁹Zr

Mice bearing xenografts (n=5) were injected with ˜300 μCi (8.4 nmol) of PEG_{40 kDa}-DFB-⁸⁹Zr and microPET/CT images were obtained at 24 h (n=2) and 48 h (n=2). The % ID/g uptake (uptake of PEG_{40 kDa}-DFB-⁸⁹Zr) in tumors was 15 and 20% at 24- and 48 h, respectively, while organs other than liver had ≤3% uptake. MicroPET/CT studies showed high accumulation of ⁸⁹Zr-DFB-PEG₄₀ in MX-1 tumors as early as 24h while accumulation in healthy tissue was nearly background. The imaging data corroborated the increased accumulation in tumor from 24 to 48h. However, there was heterogeneous uptake in the tumor, possibly suggesting necrosis of this rapidly growing tumor.

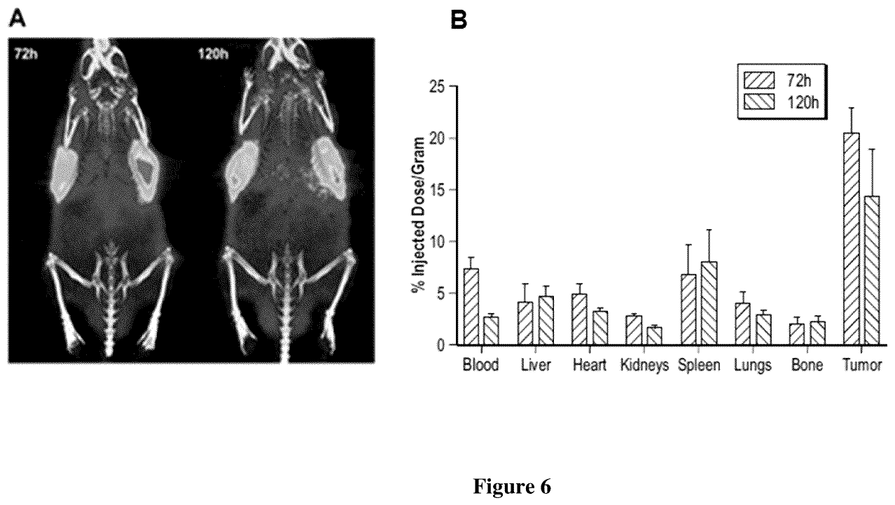

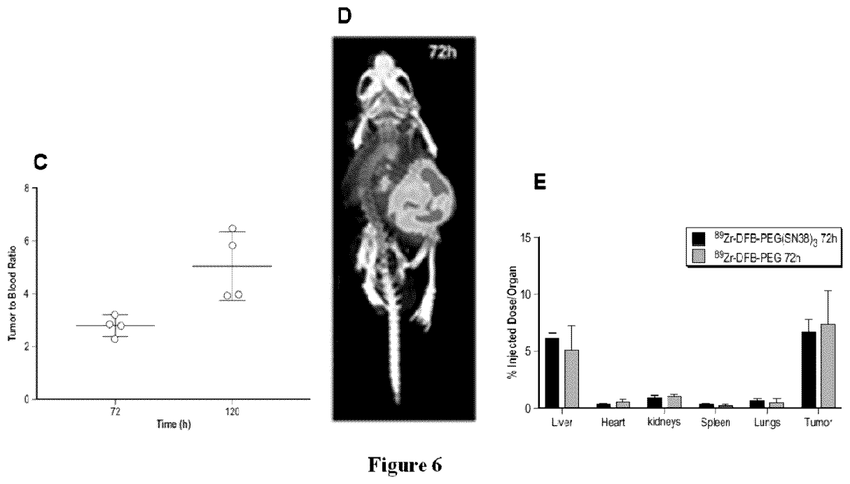

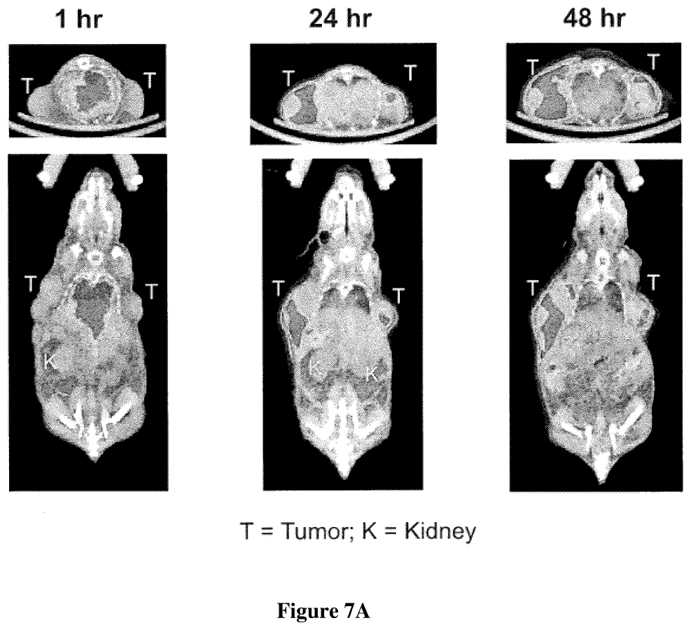

The experiment was repeated the slower growing HT-29 tumor. Given the lower tumor to blood ratios and limited clearance at early time points in MX-1 tumors (1.1±0.2 [24 h]−1.2±0.1 [48 h]) the uptake in the HT-29 tumors was studied at 72 h and 120 h. Mice (n=8) were injected with ˜160 μCi (8.4 nmol) of ⁸⁹Zr-DFB-PEG₄₀ and microPET/CT images were obtained at 72- and 120h. Mice were euthanized at 72- and 120h for ex-vivo biodistribution studies. HT-29 tumors were clearly visualized on the microPET/CT at 72h and 120h (FIG. 6A), and biodistribution studies revealed high uptake of 20.6±2.4 and 14.4±4.5% ID/g at 72 and 120h (FIG. 6B) with tumor/blood 2.8±0.4 and 5.1±1.3 at 72 and 120h, respectively (FIG. 6C). FIG. 6D is an MIP image of PEG-SN-38)₃-DFB⁸⁹Zr in a single flank tumor-bearing mouse. FIG. 6E shows biodistribution of PEG-(SN-38)₃-DFB-⁸⁹Zr (black) vs PEG-DFB-⁸⁹Zr (grey) at 72h.

In an additional study, the PEG_{40 kDa}-(DFB-⁸⁹Zr)₄ of Example 5 was injected into mice bearing HT29 tumors. Five mice were used in the study and each was injected with 250-290 Ci of the conjugate in 100 l saline. Two of the mice were imaged at one hour post injection. After 24 hours, two mice, (one that had been imaged at one hour and an additional mouse) were imaged and then sacrificed to perform distribution studies. At 48 hours, two mice were imaged (one of the mice that was imaged at one hour and one additional mouse) and these were also sacrificed along with the remaining mouse and a distribution study performed.

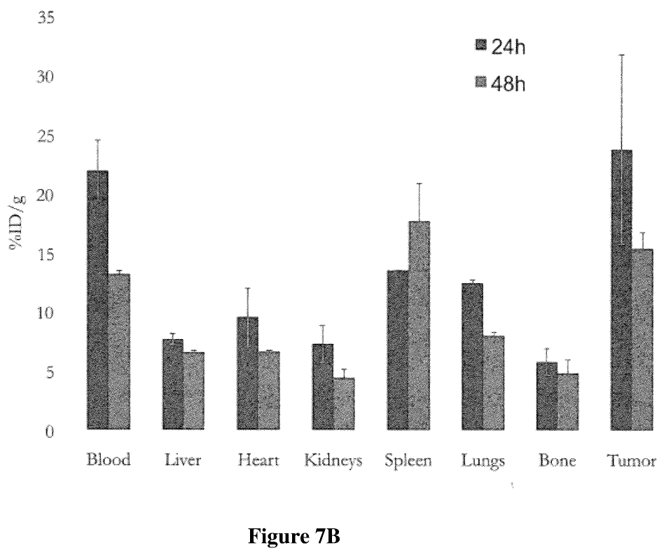

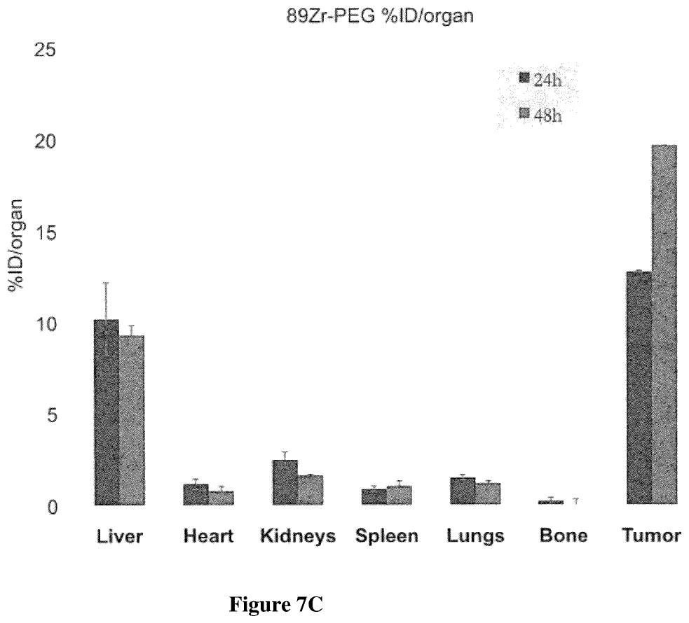

The results of these studies are shown in FIGS. 7A-7C. Shown in FIG. 7A, the label was present in the tumor at all of the times measured. As shown in FIG. 7B, the % of the injected dose (ID) per gram of individual organs was significant in most organs, although bone, spleen and tumor had the highest levels. As shown in FIG. 7C when computed as the percentage of the injected dose per organ, rather than as per gram of organ, accumulation in the tumor was dramatically higher, especially at 48 hours, as compared to other organs. Only liver showed a significant accumulation which dropped over the time period of 24-48 hours. Thus, the imaging agent confirms that the conjugate is selectively accumulated in the tumor as compared to other organs.

### Example 9

### Additional Distribution Studies

The experiments of Example 8 were repeated using 4-branched PEG_{40 kDa}-DFB-⁸⁹Zr (Example 5), 4-armed PEG_{40 kDa}-(DFB-⁸⁹Zr)₄ (Example 5), and 4-armed PEG₄₀ kDa-(DFB-⁸⁹Zr)₁(SN38)₃ (Example 6) in both MX-1 and HT-29 xenografts. PET imaging was used to measure accumulation of ⁸⁹Zr in tumor, heart, liver, and kidney at 1, 24, 48, 72, 96, and 216 h post-dose. The resulting data (expressed as decay-corrected percent of the total dose) were analyzed using a membrane-limited tissue distribution model according to the methods of Li et al., Intl. J. Nanomedicine (2012) 7: 1345-56. A compartment for the remaining tissues was included in order to match measured blood levels in the absence of more specific tissue analyses. Blood data were fit using a total clearance equal to the sum of the diffusion coefficients from blood into the organs (k, Table 2) and the elimination rate constant calculated from a plasma half-life of 20 hours.

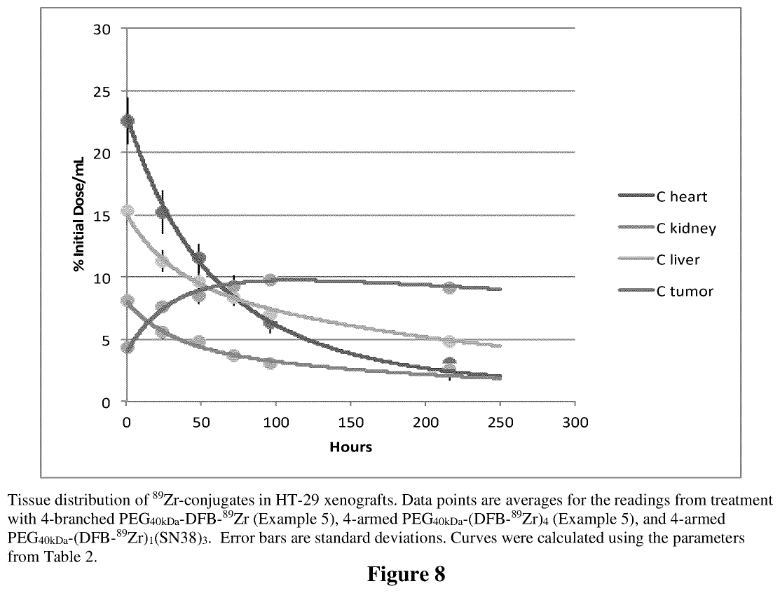

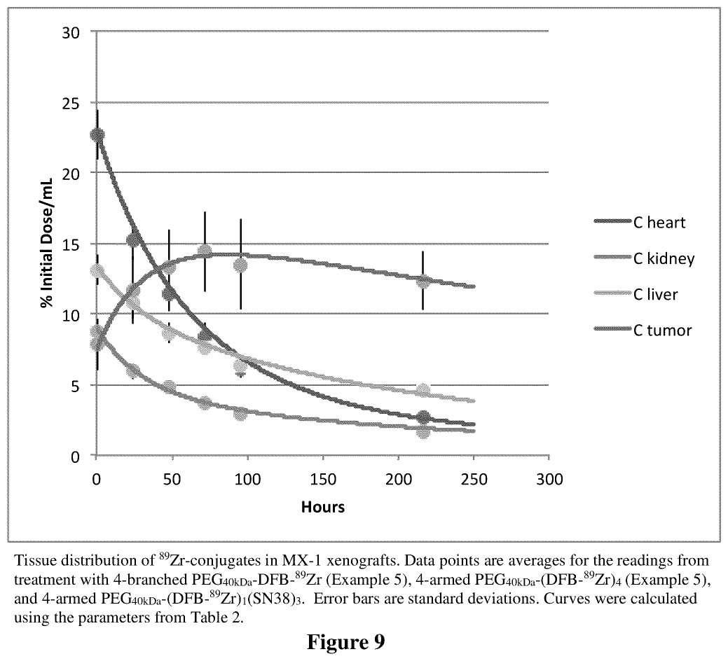

Within experimental error, all three compounds showed the same tissue distribution in a specific tumor xenograft. FIG. 8 shows the distribution of ⁸⁹Zr in HT-29 xenografts, and FIG. 9 shows the distribution of ⁸⁹Zr in MX-1 xenografts. Model parameters are given in Table 2, where R=tissue-blood partition coefficient, k=diffusion coefficient, V=tissue volume, and VVF=the vascular fraction of the tissue.

TABLE 2
Parameters for Membrane-Limited Tissue Distribution Model

| | R | k (h⁻¹) | V (mL) | VVF | k/RV |
|---|---|---|---|---|---|
| HT-29 | | | | | |
| Circulation | | | 2.8 | 1 | |
| Heart | 0.7 | 0.0015 | 0.15 | 0.23 | 0.0143 |
| Kidney | 0.6 | 0.0017 | 0.5 | 0.08 | 0.00567 |
| Liver | 1.5 | 0.013 | 1.65 | 0.15 | 0.00523 |
| Tumor | 5 | 0.0095 | 1 | 0.04 | 0.0019 |
| Body | 1 | 0.03 | 30 | 0.1 | 0.001 |
| MX-1 | | | | | |
| Circulation | | | 2.8 | 1 | |
| Heart | 0.7 | 0.0015 | 0.15 | 0.23 | 0.0143 |
| Kidney | 0.5 | 0.0015 | 0.5 | 0.09 | 0.006 |
| Liver | 1.2 | 0.012 | 1.65 | 0.134 | 0.00606 |
| Tumor | 5 | 0.0062 | 0.45 | 0.075 | 0.00276 |
| Body | 1 | 0.03 | 30 | 0.1 | 0.001 |

In both xenograft models, the ⁸⁹Zr-conjugates were observed to accumulate selectively in the tumor tissue and be retained for much longer times than in other tissues.

### Example 10

### Correlation of Biodistribution of Imaging Agent and Active Agent

In this example, the pharmacokinetics/biodistribution of the imaging agent PEG_{40 kDa}-DFB⁸⁹Zr is compared with that of PEG-SN-38.

SN-38 is the active metabolite of irinotecan (CPT-11) a widely used anticancer agent. (PEG-SN-38) is a conjugate of 4 arm PEG_{40 kDa} with 4 equivalents of SN-38, giving PEG_{40 kDa}(SN-38)₄ (Santi D V, et al., J. of Med. Chem. (2014) 57(6):2303-2314). (PEG-SN-38 is in dose escalation in Phase 1 trials and shows a long t_{1/2,β} of 6 days.)

Xenograft mice are prepared by implantation of 10⁶- to 10⁷ HT29 cells into the NSG mouse flank, and maintained until the tumors are ˜200 mm³. Time vs activity curves from microPET/CT images, blood, tumor and main organs are used to determine the accumulation/elimination rates of PEG_{40 kDa}-DFB-⁸⁹Zr in the tumor, the elimination rate from the blood and body, and the temporal activity distribution in the remainder of the mouse. Increasing concentrations of PEG_{40 kDa}-DFB-⁸⁹Zr increase the rate of accumulation, with no effect on the first-order elimination from tumors.

Varying doses of the unlabeled PEG-(SN-38)₄ conjugate are injected into animals. From preclinical toxicology studies of PEG-(SN-38), the dose to provide 50% tumor growth inhibition (TGI) in the HT-29 tumor/nude rate was 150 mg/kg. From allometric scaling, 50% TGI in the mouse should be ˜280 mg/kg. A target dose for measurable growth inhibition (e.g. ˜50% TGI) is verified.

A mixture of PEG-(SN-38)₄ and PEG-(DFB-⁸⁹Zr) is prepared that suitable for both a) achieving the therapeutic target dose, and b) monitoring tumor uptake/elimination kinetics of PEG-(DFB-⁸⁹Zr) measured by PET over 10 days, as described above. Tissues are removed to quantify biodistribution, blood sampling. Total SN-38 content of tumors is measured by HPLC of NaOH-digested tumor and blood samples at various times (Santi, et al. (supra)). The PEG-(SN-38)₄/PEG-(DFB-⁸⁹Zr) ratio is determined at various time points to verify either an identity of drug/isotope of the ratio vs time or other relationship of tumor uptake of two components.

The % ID/g tumor of PEG-(DFB-⁸⁹Zr) that corresponds to a therapeutic dose of PEG-(SN-38)₄ is established. High-uptake tumors are identified that accumulate sufficient PEG-(SN-38)₄ to achieve a therapeutic dose.

Thus, the subjects who will benefit from an EPR effect of a conjugated SN-38 are identified by an initial administration of the imaging agent of the invention.

### Example 11

### Efficacy of PLX038A

The SN38 conjugate designated PLX038A in Example 1 and abbreviated here as PEG-SN38 is used in this Example.

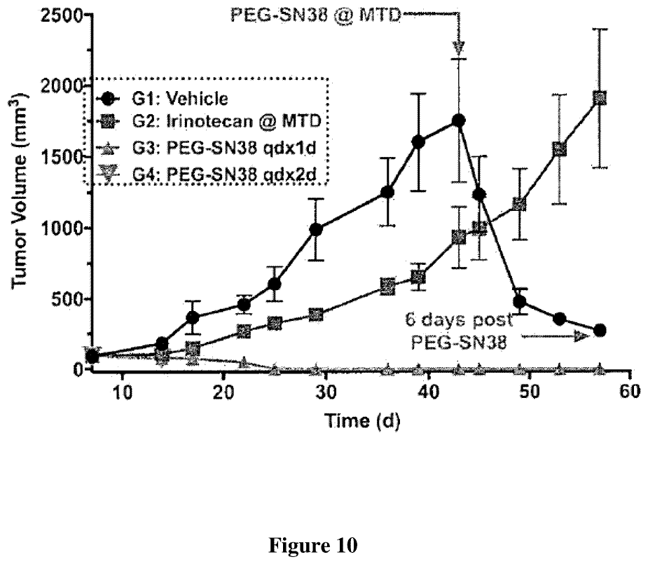

Four groups of mice having 5 mice in each group bearing MX-1 tumor xenographs were injected with vehicle or with a single dose of either vehicle, 137 mole/kg irinotecan (0.137/g or ˜4 μmole per mouse) or with 120 mole/kg PEG-SN38 qdx×1d (single dose). Tumor volume was measured as a function of time. At 42 days, the group that received vehicle was treated with 120 mole/kg of PEG-SN28. The results are shown in FIG. 10.

As shown, MX-1 tumor growth in the mice injected with vehicle continued apace, reaching 1200 mm³ after 4 weeks, for the initial 42 days until the PEG-SN38 was injected whereupon the tumor volume declined dramatically. Dosage at time 0 with PEG-SN38 immediately eliminated the tumor. Irinotecan, while having some effect, was only somewhat better than vehicle—after 4 weeks these tumors reached 600 mm³.

Further, for mice with untreated tumors that showed tumor growth even as large as 1.7 cm³, a single MTD dose of PEGSN38 shrank these tumors.

These results demonstrate that PEG-SN38 is highly effective for treating solid tumors and that the findings with the imaging agent in Example 8 are consistent with this result.

### Example 12

### Synergistic Effect of PLX038A and PARP Inhibitor Talazoparib (Designated BMN673 or TLZ)

Preparation of murine MX-1 xenografts: The MX-1 cell line was obtained from Charles River Labs (Frederick, Maryland). Ovejera A A et al. Ann Clin Lab Sci (1978) 8:50-6. Cells were cultured in RPMI-1640, 10% FBS and 1% 2 mM L-glutamine at 37° C. in 95% air/5% CO₂ atmosphere.

Female NCr nude mice (NCrTac:NCr-Foxn1^{nu}; ˜6-7 weeks old) from Taconic Bioscience (Cambridge City, Indiana) were housed at the UCSF Preclinical Therapeutics Core vivarium (San Francisco, California). All animal studies were carried out in accordance with UCSF Institutional Animal Care and Use Committee. Tumor xenografts were established by subcutaneous injection with MX-1 tumor cells (2×10⁶ cells in 100 μl of serum free medium mixed 1:1 with Matrigel) into the right flank of female NCr nude mice. When tumor xenografts reached 1000-1500 mm³ in donor mice, they were resected, cut into even-size fragments (˜2.5×2.5×2.5 mm in size), embedded in Matrigel and re-implanted via subcutaneous trocar implantation in receiver mice. Morton C L, Houghton P J. Nat Protoc. (2007) 2:247-50.

Dosing and tumor volume measurements: Solutions of PLX038A (1.02 mM SN38; 0.26 mM PLX038A conjugate) were prepared in pH 5 isotonic acetate and sterile filtered (0.2 m) before use. Solutions of BMN673 (52 M) were prepared in 10% dimethylacetamide/5% Solutol HS15/85% 1×PBS and were sterile filtered (0.2 m) before use.

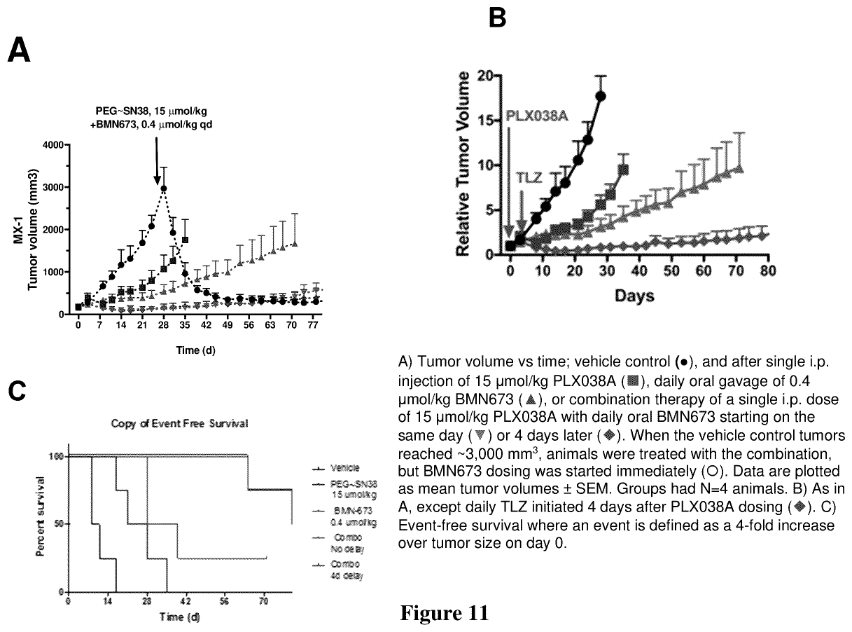

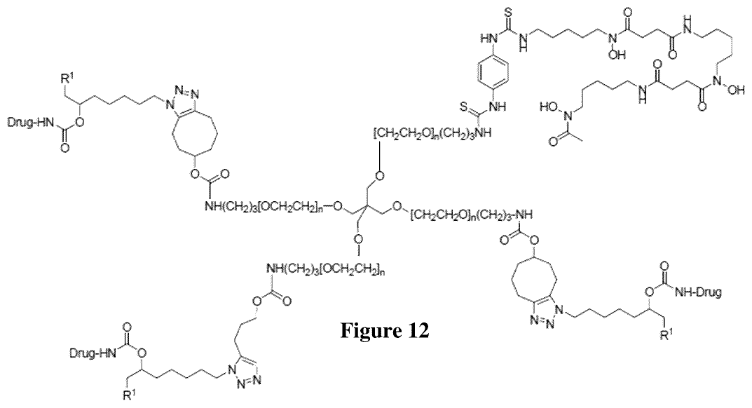

Groups (N=4-5/group) were dosed when the group average reached 100-200 mm³ in size. Mice received vehicle, a single dose of PLX038A (14.7 mL/kg i.p., 15 mol/kg), daily doses of BMN673 (7.72 mL/kg p.o., 0.4 mol/kg), or a combination of PLX038A and BMN673 at the same doses. For groups receiving the combination, daily BMN673 dosing began on the same day (FIG. 11A) or after a 4-day delay (FIG. 11B) after dosing PLX038A. Tumor volumes (caliper measurement: 0.5×(length×width²)) and body weights were measured twice weekly. When vehicle control tumors reached ˜3000 mm³ in size, mice were treated with the combination of a single dose of PLX038A (15 mol/kg) and daily BMN673 (0.4 mol/kg) combination with no delay between dosing (FIG. 11A).

As shown in FIGS. 11A and 11B, administration of PLX038A to mice bearing MX-1 tumors at 15 μmol/kg in combination with daily doses of Talazoparib at 0.4 μmol/kg provides a synergistic effect as compared to either of these drugs alone. This was true whether daily dosage with TLZ began at the same time as PLX038A or 4 days later. A single combination administered to control immediately reduced tumor volume (FIG. 11A).

As shown in FIG. 11C, event-free survival was enhanced synergistically with the combination vs PLX038A and TLZ individually.

1-21. (canceled)

22. An imaging agent of the formula (1) 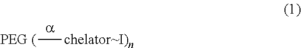
wherein PEG represents a multi-armed polyethylene glycol of 40-60 kD, comprising 4 arms;
chelator represents a desferrioxamine or a plur-hydroxypyridinone multidentate;
I is a radioisotope suitable for positron emission tomography (PET); 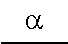
is a covalent connector;
˜ indicates sequestration of I in the chelator; and
n is an integer of 1 up to the number of arms of said PEG.

23. The imaging agent of claim 22 wherein I is ⁸⁹Zr, ⁹⁴Tc, ¹⁰¹In, ⁸¹Rb, ⁶⁶Ga, ⁶⁴Cu, ⁶²Zn, ⁶¹Cu or ⁵²Fe.

24. A method to monitor accumulation of the imaging agent of claim 22 in a tumor which method comprises administering said imaging agent and detecting the location of said imaging agent by PET.

25. A method to assess pharmacokinetics of a conjugate of a drug and its accumulation in a tumor, which method comprises matching the size and shape of the conjugate of said drug to the size and shape of the imaging agent of claim 22, administering said imaging agent to a subject bearing the tumor, and monitoring the accumulation of said agent in the tumor by PET.

26. A kit that comprises the imaging agent of claim 22 and a drug conjugate.

27. A method to identify a subject having an undesirable tissue mass likely to benefit from treatment with a drug modified to exhibit an enhanced permeability and retention (EPR) effect, which comprises administering the imaging agent of claim 22 to a candidate subject; and
monitoring the distribution of the imaging agent in the subject,
whereby a subject that accumulates said imaging agent in said undesirable tissue mass is identified as a subject that will benefit from such treatment.

28. The method of claim 27 which further comprises determining the presence or absence of a mutation in a gene that encodes a protein that participates in effecting DNA repair, wherein the presence of said mutation in the subject identifies the subject as having said undesirable tissue mass.

29. The method of claim 28 wherein the gene is BRCA1, BRCA2, ATM or ATR.

30-33. (canceled)

34. The imaging agent of claim 22 wherein chelator is desferrioxamine-B.

35. The imaging agent of claim 22 wherein 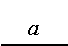
is a direct bond linkage.

36. The imaging agent of claim 22 wherein PEG is a four armed polyethylene glycol of approximately 40 kD, and n is 1-4.

37. The imaging agent of claim 22 wherein:
I is ⁸⁹Zr, ⁹⁴Tc, ¹⁰¹In, ⁸¹Rb, ⁶⁶Ga, ⁶⁴Cu, ⁶²Zn, ⁶¹Cu or ⁵²Fe;
PEG is a four armed polyethylene glycol of approximately 40 kD, and n is 1-4; and
chelator is desferrioxamine-B.

38. The imaging agent of claim 22 wherein:
I is ⁸⁹Zr, ⁹⁴Tc, ¹⁰¹In, ⁸¹Rb, ⁶⁶Ga, ⁶⁴Cu, ⁶²Zn, ⁶¹Cu or ⁵²Fe;
PEG is a four armed polyethylene glycol of approximately 40 kD, and n is 1-4;
chelator is desferrioxamine-B; and 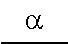
is a direct bond linkage.

39. The imaging agent of claim 22 wherein the imaging agent has a hydrodynamic radius of 5-50 nm.
